SEQUENCE LISTING (164 sequences)

SEQ ID NO: 1 - protein, 30 aa, Homo sapiens
ALFSVLNYERARRPGLLGASVLGLDDIHRA

SEQ ID NO: 2 - protein, 15 aa, Homo sapiens
SVLNYERARRPGLLG

SEQ ID NO: 3 - protein, 10 aa, Homo sapiens
GLLGASVLGL

SEQ ID NO: 4 - protein, 10 aa, Homo sapiens
ALLTSRLRFI

SEQ ID NO: 5 - protein, 9 aa, Homo sapiens
RPALLTSRL

SEQ ID NO: 6 - protein, 9 aa, Homo sapiens
RLTSRVKAL

SEQ ID NO: 7 - protein, 15 aa, Homo sapiens
RTFVLRVRAQDPPPE

SEQ ID NO: 8 - protein, 15 aa, Homo sapiens
RLTSRVKALFSVLNY

SEQ ID NO: 9 - protein, 15 aa, Homo sapiens
AERLTSRVKALFSVL

SEQ ID NO: 10 - protein, 9 aa, Homo sapiens
YERARRPGL

SEQ ID NO: 11 - protein, 9 aa, Homo sapiens
LLGASVLGL

SEQ ID NO: 12 - protein, 10 aa, Homo sapiens
VLGLDDIHRA

SEQ ID NO: 13 - protein, 10 aa, Homo sapiens
GASVLGLDDI

SEQ ID NO: 14 - protein, 10 aa, Homo sapiens
ALFSVLNYER

SEQ ID NO: 15 - protein, 10 aa, Homo sapiens
SVLNYERARR

SEQ ID NO: 16 - protein, 10 aa, Homo sapiens
SVLGLDDIHR

SEQ ID NO: 17 - protein, 10 aa, Homo sapiens
FSVLNYERAR

SEQ ID NO: 18 - protein, 10 aa, Homo sapiens
NYERARRPGL

SEQ ID NO: 19 - protein, 10 aa, Homo sapiens
YERARRPGLL

SEQ ID NO: 20 - protein, 10 aa, Homo sapiens
RPGLLGASVL

SEQ ID NO: 21 - protein, 9 aa, Homo sapiens
ERARRPGLL

SEQ ID NO: 22 - protein, 9 aa, Homo sapiens
VLGLDDIHR

SEQ ID NO: 23 - protein, 9 aa, Homo sapiens
SVLNYERAR

SEQ ID NO: 24 - protein, 9 aa, Homo sapiens
VLNYERARR

SEQ ID NO: 25 - protein, 9 aa, Homo sapiens
ARRPGLLGA

SEQ ID NO: 26 - protein, 9 aa, Homo sapiens
PGLLGASVL

SEQ ID NO: 27 - protein, 9 aa, Homo sapiens
RRPGLLGAS

SEQ ID NO: 28 - protein, 9 aa, Homo sapiens
ALFSVLNYE

SEQ ID NO: 29 - protein, 9 aa, Homo sapiens
GLLGASVLG

SEQ ID NO: 30 - protein, 9 aa, Homo sapiens
SVLGLDDIH

SEQ ID NO: 31 - protein, 8 aa, Homo sapiens
ERARRPGL

SEQ ID NO: 32 - protein, 8 aa, Homo sapiens
ARRPGLLG

SEQ ID NO: 33 - protein, 8 aa, Homo sapiens
GLLGASVL

SEQ ID NO: 34 - protein, 8 aa, Homo sapiens
RARRPGLL

SEQ ID NO: 35 - protein, 8 aa, Homo sapiens
LGASVLGL

SEQ ID NO: 36 - protein, 15 aa, Homo sapiens
VTETTFQKNRLFFYR

SEQ ID NO: 37 - protein, 15 aa, Homo sapiens
LFFYRKSVWSKLQSI

SEQ ID NO: 38 - protein, 15 aa, Homo sapiens
KLQSIGIRQHLKRVQ

SEQ ID NO: 39 - protein, 15 aa, Homo sapiens
EAEVRQHREARPALL

SEQ ID NO: 40 - protein, 15 aa, Homo sapiens
RPALLTSRLRFIPKP

SEQ ID NO: 41 - protein, 15 aa, Homo sapiens
FIPKPDGLRPIVNMD

SEQ ID NO: 42 - protein, 15 aa, Homo sapiens
RTFRREKRAERLTSR

SEQ ID NO: 43 - protein, 15 aa, Homo sapiens
LDDIHRAWRTFVLRV

SEQ ID NO: 44 - protein, 15 aa, Homo sapiens
FVLRVRAQDPPPELY

SEQ ID NO: 45 - protein, 15 aa, Homo sapiens
PQDRLTEVIASIIKP

SEQ ID NO: 46 - protein, 15 aa, Homo sapiens
KSVWSKLQSIGIRQH

SEQ ID NO: 47 - protein, 16 aa, Homo sapiens
LKRVQLRELSEAEVRQ

SEQ ID NO: 48 - protein, 20 aa, Homo sapiens
MPRAPRCRAVRSLLRSHYRE

SEQ ID NO: 49 - protein, 20 aa, Homo sapiens
VLPLATFVRRLGPQGWRLVQ

SEQ ID NO: 50 - protein, 20 aa, Homo sapiens
RGDPAAFRALVAQCLVCVPW

SEQ ID NO: 51 - protein, 20 aa, Homo sapiens
DARPPPAAPSFRQVSCLKEL

SEQ ID NO: 52 - protein, 20 aa, Homo sapiens
VARVLQRLCERGAKNVLAFG

SEQ ID NO: 53 - protein, 20 aa, Homo sapiens
FALLDGARGGPPEAFTTSVR

SEQ ID NO: 54 - protein, 20 aa, Homo sapiens
SYLPNTVTDALRGSGAWGLL

SEQ ID NO: 55 - protein, 20 aa, Homo sapiens
LRRVGDDVLVHLLARCALFV

SEQ ID NO: 56 - protein, 20 aa, Homo sapiens
LVAPSCAYQVCGPPLYQLGA

SEQ ID NO: 57 - protein, 20 aa, Homo sapiens
ATQARPPPHASGPRRRLGCE

SEQ ID NO: 58 - protein, 20 aa, Homo sapiens
RAWNHSVREAGVPLGLPAPG

SEQ ID NO: 59 - protein, 20 aa, Homo sapiens
ARRRGGSASRSLPLPKRPRR

SEQ ID NO: 60 - protein, 20 aa, Homo sapiens
GAAPEPERTPVGQGSWAHPG

SEQ ID NO: 61 - protein, 20 aa, Homo sapiens
RTRGPSDRGFCVVSPARPAE

SEQ ID NO: 62 - protein, 20 aa, Homo sapiens
EATSLEGALSGTRHSHPSVG

SEQ ID NO: 63 - protein, 20 aa, Homo sapiens
RQHHAGPPSTSRPPRPWDTP

SEQ ID NO: 64 - protein, 20 aa, Homo sapiens
CPPVYAETKHFLYSSGDKEQ

SEQ ID NO: 65 - protein, 20 aa, Homo sapiens
LRPSFLLSSLRPSLTGARRL

SEQ ID NO: 66 - protein, 20 aa, Homo sapiens
VETIFLGSRPWMPGTPRRLP

SEQ ID NO: 67 - protein, 20 aa, Homo sapiens
RLPQRYWQMRPLFLELLGNH

SEQ ID NO: 68 - protein, 20 aa, Homo sapiens
AQCPYGVLLKTHCPLRAAVT

SEQ ID NO: 69 - protein, 20 aa, Homo sapiens
PAAGVCAREKPQGSVAAPEE

SEQ ID NO: 70 - protein, 20 aa, Homo sapiens
EDTDPRRLVQLLRQHSSPWQ

SEQ ID NO: 71 - protein, 20 aa, Homo sapiens
RHNERRFLRNTKKFISLGKH

SEQ ID NO: 72 - protein, 20 aa, Homo sapiens
AKLSLQELTWKMSVRDCAWL

SEQ ID NO: 73 - protein, 20 aa, Homo sapiens
RRSPGVGCVPAAEHRLREEI

SEQ ID NO: 74 - protein, 20 aa, Homo sapiens
LAKFLHWLMSVYVVELLRSF

SEQ ID NO: 75 - protein, 20 aa, Homo sapiens
FYVTETTFQKNRLFFYRKSV

SEQ ID NO: 76 - protein, 20 aa, Homo sapiens
WSKLQSIGIRQHLKRVQLRE

SEQ ID NO: 77 - protein, 20 aa, Homo sapiens
LSEAEVRQHREARPALLTSR

SEQ ID NO: 78 - protein, 20 aa, Homo sapiens
LRFIPKPDGLRPIVNMDYVV

SEQ ID NO: 79 - protein, 20 aa, Homo sapiens
GARTFRREKRAERLTSRVKA

SEQ ID NO: 80 - protein, 20 aa, Homo sapiens
LFSVLNYERARRPGLLGASV

SEQ ID NO: 81 - protein, 20 aa, Homo sapiens
DPPPELYFVKVDVTGAYDTI

SEQ ID NO: 82 - protein, 20 aa, Homo sapiens
PQDRLTEVIASIIKPQNTYC

SEQ ID NO: 83 - protein, 20 aa, Homo sapiens
VRRYAVVQKAAHGHVRKAFK

SEQ ID NO: 84 - protein, 20 aa, Homo sapiens
SHVSTLTDLQPYMRQFVAHL

SEQ ID NO: 85 - protein, 20 aa, Homo sapiens
QETSPLRDAVVIEQSSSLNE

SEQ ID NO: 86 - protein, 20 aa, Homo sapiens
ASSGLFDVFLRFMCHHAVRI

SEQ ID NO: 87 - protein, 20 aa, Homo sapiens
RGKSYVQCQGIPQGSILSTL

SEQ ID NO: 88 - protein, 20 aa, Homo sapiens
LCSLCYGDMENKLFAGIRRD

SEQ ID NO: 89 - protein, 20 aa, Homo sapiens
GLLLRLVDDFLLVTPHLTHA

SEQ ID NO: 90 - protein, 20 aa, Homo sapiens
KTFLRTLVRGVPEYGCVVNL

SEQ ID NO: 91 - protein, 20 aa, Homo sapiens
RKTVVNFPVEDEALGGTAFV

SEQ ID NO: 92 - protein, 20 aa, Homo sapiens
QMPAHGLFPWCGLLLDTRTL

SEQ ID NO: 93 - protein, 20 aa, Homo sapiens
EVQSDYSSYARTSIRASLTF

SEQ ID NO: 94 - protein, 20 aa, Homo sapiens
KCHSLFLDLQVNSLQTVCTN

SEQ ID NO: 95 - protein, 20 aa, Homo sapiens
IYKILLLQAYRFHACVLQLP

SEQ ID NO: 96 - protein, 20 aa, Homo sapiens
FHQQVWKNPTFFLRVISDTA

SEQ ID NO: 97 - protein, 20 aa, Homo sapiens
SLCYSILKAKNAGMSLGAKG

SEQ ID NO: 98 - protein, 20 aa, Homo sapiens
AAGPLPSEAVQWLCHQAFLL

SEQ ID NO: 99 - protein, 20 aa, Homo sapiens
KLTRHRVTYVPLLGSLRTAQ

SEQ ID NO: 100 - protein, 20 aa, Homo sapiens
TQLSRKLPGTTLTALEAAAN

SEQ ID NO: 101 - protein, 12 aa, Homo sapiens
PALPSDFKTILD

SEQ ID NO: 102 - protein, 10 aa, Homo sapiens
MPRAPRCRAV

SEQ ID NO: 103 - protein, 20 aa, Homo sapiens
RSLLRSHYREVLPLATFVRR

SEQ ID NO: 104 - protein, 20 aa, Homo sapiens
LGPQGWRLVQRGDPAAFRAL

SEQ ID NO: 105 - protein, 20 aa, Homo sapiens
VAQCLVCVPWDARPPPAAPS

SEQ ID NO: 106 - protein, 20 aa, Homo sapiens
FRQVSCLKELVARVLQRLCE

SEQ ID NO: 107 - protein, 20 aa, Homo sapiens
RGAKNVLAFGFALLDGARGG

SEQ ID NO: 108 - protein, 20 aa, Homo sapiens
PPEAFTTSVRSYLPNTVTDA

SEQ ID NO: 109 - protein, 20 aa, Homo sapiens
LRGSGAWGLLLRRVGDDVLV

SEQ ID NO: 110 - protein, 20 aa, Homo sapiens
HLLARCALFVLVAPSCAYQV

SEQ ID NO: 111 - protein, 20 aa, Homo sapiens
CGPPLYQLGAATQARPPPHA

SEQ ID NO: 112 - protein, 20 aa, Homo sapiens
SGPRRRLGCERAWNHSVREA

SEQ ID NO: 113 - protein, 20 aa, Homo sapiens
GVPLGLPAPGARRRGGSASR

SEQ ID NO: 114 - protein, 20 aa, Homo sapiens
SLPLPKRPRRGAAPEPERTP

SEQ ID NO: 115 - protein, 20 aa, Homo sapiens
VGQGSWAHPGRTRGPSDRGF

SEQ ID NO: 116 - protein, 20 aa, Homo sapiens
CVVSPARPAEEATSLEGALS

SEQ ID NO: 117 - protein, 20 aa, Homo sapiens
GTRHSHPSVGRQHHAGPPST

SEQ ID NO: 118 - protein, 20 aa, Homo sapiens
SRPPRPWDTPCPPVYAETKH

SEQ ID NO: 119 - protein, 20 aa, Homo sapiens
FLYSSGDKEQLRPSFLLSSL

SEQ ID NO: 120 - protein, 20 aa, Homo sapiens
RPSLTGARRLVETIFLGSRP

SEQ ID NO: 121 - protein, 20 aa, Homo sapiens
WMPGTPRRLPRLPQRYWQMR

SEQ ID NO: 122 - protein, 20 aa, Homo sapiens
PLFLELLGNHAQCPYGVLLK

SEQ ID NO: 123 - protein, 20 aa, Homo sapiens
THCPLRAAVTPAAGVCAREK

SEQ ID NO: 124 - protein, 20 aa, Homo sapiens
PQGSVAAPEEEDTDPRRLVQ

SEQ ID NO: 125 - protein, 20 aa, Homo sapiens
LLRQHSSPWQVYGFVRACLR

SEQ ID NO: 126 - protein, 20 aa, Homo sapiens
RLVPPGLWGSRHNERRFLRN

SEQ ID NO: 127 - protein, 20 aa, Homo sapiens
TKKFISLGKHAKLSLQELTW

SEQ ID NO: 128 - protein, 20 aa, Homo sapiens
KMSVRDCAWLRRSPGVGCVP

SEQ ID NO: 129 - protein, 20 aa, Homo sapiens
AAEHRLREEILAKFLHWLMS

SEQ ID NO: 130 - protein, 20 aa, Homo sapiens
VYVVELLRSFFYVTETTFQK

SEQ ID NO: 131 - protein, 20 aa, Homo sapiens
NRLFFYRKSVWSKLQSIGIR

SEQ ID NO: 132 - protein, 20 aa, Homo sapiens
QHLKRVQLRELSEAEVRQHR

SEQ ID NO: 133 - protein, 20 aa, Homo sapiens
EARPALLTSRLRFIPKPDGL

SEQ ID NO: 134 - protein, 20 aa, Homo sapiens
RPIVNMDYVVGARTFRREKR

SEQ ID NO: 135 - protein, 20 aa, Homo sapiens
AERLTSRVKALFSVLNYERA

SEQ ID NO: 136 - protein, 20 aa, Homo sapiens
RRPGLLGASVLGLDDIHRAW

SEQ ID NO: 137 - protein, 20 aa, Homo sapiens
RTFVLRVRAQDPPPELYFVK

SEQ ID NO: 138 - protein, 20 aa, Homo sapiens
VDVTGAYDTIPQDRLTEVIA

SEQ ID NO: 139 - protein, 20 aa, Homo sapiens
AHGHVRKAFKSHVSTLTDLQ

SEQ ID NO: 140 - protein, 20 aa, Homo sapiens
PYMRQFVAHLQETSPLRDAV

SEQ ID NO: 141 - protein, 20 aa, Homo sapiens
VIEQSSSLNEASSGLFDVFL

SEQ ID NO: 142 - protein, 20 aa, Homo sapiens
RFMCHHAVRIRGKSYVQCQG

SEQ ID NO: 143 - protein, 20 aa, Homo sapiens
IPQGSILSTLLCSLCYGDME

SEQ ID NO: 144 - protein, 20 aa, Homo sapiens
NKLFAGIRRDGLLLRLVDDF

SEQ ID NO: 145 - protein, 20 aa, Homo sapiens
LLVTPHLTHAKTFLRTLVRG

SEQ ID NO: 146 - protein, 20 aa, Homo sapiens
VPEYGCVVNLRKTVVNFPVE

SEQ ID NO: 147 - protein, 20 aa, Homo sapiens
DEALGGTAFVQMPAHGLFPW

SEQ ID NO: 148 - protein, 20 aa, Homo sapiens
CGLLLDTRTLEVQSDYSSYA

SEQ ID NO: 149 - protein, 20 aa, Homo sapiens
RRKLFGVLRLKCHSLFLDLQ

SEQ ID NO: 150 - protein, 20 aa, Homo sapiens
VNSLQTVCTNIYKILLLQAY

SEQ ID NO: 151 - protein, 20 aa, Homo sapiens
RFHACVLQLPFHQQVWKNPT

SEQ ID NO: 152 - protein, 20 aa, Homo sapiens
FFLRVISDTASLCYSILKAK

SEQ ID NO: 153 - protein, 20 aa, Homo sapiens
NAGMSLGAKGAAGPLPSEAV

SEQ ID NO: 154 - protein, 20 aa, Homo sapiens
QWLCHQAFLLKLTRHRVTYV

SEQ ID NO: 155 - protein, 20 aa, Homo sapiens
PLLGSLRTAQTQLSRKLPGT

SEQ ID NO: 156 - protein, 22 aa, Homo sapiens
TLTALEAAANPALPSDFKTILD

SEQ ID NO: 157 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 158 - protein, 239 aa, synthetic construct
  REGION 1..239: note = Green Fluorescent Protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 159 - DNA, 720 bp, synthetic construct
  misc_feature 1..720: note = Green Fluorescent Protein
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 160 - DNA, 21 bp, synthetic construct
  misc_feature 1..19: note = RNA
  source 1..21: mol_type = other DNA
  misc_feature 1..21: note = siRNA sense
  modified_base 20..21: mod_base = OTHER; note = deoxythymidine (dT)
cttacgctgagtacttcgann

SEQ ID NO: 161 - DNA, 21 bp, synthetic construct
  misc_feature 1..19: note = RNA
  source 1..21: mol_type = other DNA
  misc_feature 1..21: note = siRNA antisense
  modified_base 20..21: mod_base = OTHER; note = deoxythymidine (dT)
tcgaagtactcagcgtaagnn

SEQ ID NO: 162 - DNA, 21 bp, synthetic construct
  misc_feature 1..19: note = RNA
  source 1..21: mol_type = other DNA
  misc_feature 1..21: note = siCont sense
  modified_base 20..21: mod_base = OTHER; note = deoxythymidine (dT)
gcacctataacaacggtagnn

SEQ ID NO: 163 - DNA, 21 bp, synthetic construct
  misc_feature 1..19: note = RNA
  source 1..21: mol_type = other DNA
  modified_base 20..21: mod_base = OTHER; note = deoxythymidine (dT)
  misc_feature 1..21: note = siCont antisense
ctaccgttgttataggtgcnn

SEQ ID NO: 164 - protein, 16 aa, Homo sapiens
EARPALLTSRLRFIPK

(19) United States
(12) Patent Application Publication
(10) Pub. No.: US 2024/0084273 A1
(43) Pub. Date: Mar. 14, 2024

(54) CELL PENETRATING PEPTIDE, CONJUGATE COMPRISING SAME, AND COMPOSITION COMPRISING CONJUGATE

(71) Applicants: Sang Jae Kim, Seoul (KR); GemVax & KAEL CO., LTD., Daejeon (KR)

(72) Inventor: Sang Jae KIM, Seoul (KR)

(21) Appl. No.: 18/451,280

(22) Filed: Aug. 17, 2023

Related U.S. Application Data
(62) Division of application No. 17/032,781, filed on Sep. 25, 2020, now Pat. No. 11,773,381.
Application No. 17/032,781 is a division of application No. 15/869,518, filed on Jan. 12, 2018, now Pat. No. 10,822,595.
Application No. 15/869,518 is a division of application No. 14/429,637, filed on Mar. 19, 2015, now Pat. No. 9,902,945.

(30) Foreign Application Priority Data
Sep. 19, 2012 (KR) 10-2012-0104144
Sep. 19, 2012 (KR) 10-2012-0104173
Sep. 28, 2012 (KR) 10-2012-0109207
Sep. 28, 2012 (KR) 10-2012-0109216
Feb. 18, 2013 (KR) 10-2013-0017169

(51) Int. Cl. C12N 9/12; A23L 33/18; A61K 8/64; A61K 8/66; A61K 47/64; A61K 49/00; A61Q 19/00; C07K 7/08; C07K 14/00; G01N 33/58

(52) U.S. Cl. CPC C12N 9/1276; A23L 33/18; A61K 8/64; A61K 8/66; A61K 47/64; A61K 49/0002; A61K 49/0043; A61K 49/0056; A61Q 19/00; C07K 7/08; C07K 14/00; G01N 33/582; A61K 38/00; C07K 2319/10

(57) ABSTRACT

The present invention relates to a conjugate of cell penetrating peptide and an active ingredient; and its use. Specifically, a conjugate including a cell penetrating peptide which is a peptide comprising any one amino acid sequence of SEQ ID NO: 1 to SEQ ID NO: 156, a fragment of any one sequence of SEQ ID NO: 1 to SEQ ID NO: 156, or a peptide having above 80% homology with the above-mentioned sequence; and a composition comprising the same are disclosed.

### SEQUENCE LISTING

The instant application contains a Sequence Listing which has been submitted electronically in XML format and is hereby incorporated by reference in its entirety. Said XML copy, created on Dec. 4, 2023, is named 51553-011004_Sequence_Listing_12_4_23 and is 151.552 bytes in size.

### FIELD OF THE INVENTION

The present invention relates to cell penetrating peptides derived from human telomerase reverse transcriptase (hTERT) enzyme, conjugates of the cell penetrating peptides, active ingredients, and compositions comprising the conjugate.

### BACKGROUND

Although Low-molecular weight substances, nucleic acids, proteins, nano-particles, etc, have great potentials as therapeutic substances at a molecular level, their uses are limited due to the incompetence to penetrate into tissues and cell membrane.. The development of a system to deliver such substances into the cell has been the active area of research over the last two decades. transport the substances inside the cell has been a conversation topic in a treatment of molecular method. Low-molecular weight substances, nucleic acids or nano-particles were transported inside the cell by several reagents, electroporation or heatshock. However, it was difficult to find an adequate method of delivery of proteins inside the cell without disrupting the activity and integrity of proteins. In 1980s, in the research conducted on studying the cell penetrating capacity of HIV, it was found that HIV-TAT protein consisting of specific 11 amino acids play an important role in a process of transportation inside the cell. Thus, in 1990s, studies on finding the right method of transporting proteins inside the cell has been the intense area of research.

Telomere is known as a repetitive sequence of genetic material found at the ends of chromosomes that prevent chromosomes from damage or merging onto other chromosomes. The length of the telomere is shortened at each cell division, and after a certain number of cell division, the telomere length is extremely shortened to the extent in which the cell stops dividing and dies. On the other hand, the elongation of telomeres is known to extend the life span of a cell. For an example, cancer cells excrete an enzyme called telomerase, which prevents shortening of telomeres, thus resulting in proliferation of cancer cells.

The objective of this invention is to provide a novel peptide.

Another objective of present invention is to provide the polynucleotide that codes the novel peptide.

Another objective of present invention is to provide a cell penetrating peptide.

Another objective of present invention is to provide a useful peptide as a carrier of the active ingredient in a cell.

Another objective of present invention is to provide a conjugate that an active ingredient and cell penetrating peptide are conjugated.

Another objective of present invention is to provide a composition comprising a conjugate of an active ingredient and cell penetrating peptide.

Another objective of present invention is to provide a pharmaceutical composition comprising a conjugate of an active ingredient and cell penetrating peptide.

Another objective of present invention is to provide a functional cosmetic composition comprising a conjugate of an active ingredient and cell penetrating peptide.

Another objective of present invention is to provide a health food composition comprising a conjugate of an active ingredient and cell penetrating peptide.

Another objective of present invention is to provide a contrast agent comprising a conjugate of an active ingredient and cell penetrating peptide.

### SUMMARY OF THE INVENTION

The conjugate according to the one embodiment of the present invention may be a conjugate of cell penetrating carrier peptide and active ingredients, wherein the carrier peptide is the peptide comprising any one amino acid sequence of SEQ ID NO: 1 to SEQ ID NO: 156, the peptide having above 80% homology of amino acid sequence with above-mentioned sequences, or the fragment of the above-mentioned peptides, and wherein the peptide having above 80% homology of amino acid sequence with above-mentioned sequences and the fragment of the same are the peptides that maintain cell penetrating ability of any one amino acid sequences of 1 to SEQ ID NO: 156.

According to another embodiment of the conjugate in the present invention, the fragment may be made of 3 or more amino acids.

According to another embodiment of the conjugate in the present invention, the carrier peptide may be made of 30 or less amino acids.

According to another embodiment of the conjugate in the present invention, the above-mentioned carrier peptide may be the peptide having any one or more amino acid sequences of SEQ ID NO: 1 to SEQ ID NO: 156.

According to another embodiment of the conjugate in the present invention, which comprises any one amino acid sequence selected from the group consisting of: SEQ ID NO: 9, SEQ ID NO: 37, SEQ ID NO: 43, SEQ ID NO: 48, SEQ ID NO: 52, SEQ ID NO: 55, SEQ ID NO: 90, SEQ ID NO: 92 and SEQ ID NO: 121.

The contrast agent according to the one embodiment of the present invention may comprise any one conjugate above-mentioned.

The contrast agent according to the one embodiment of the present invention may be for contrasting a cell.

According to another embodiment of the contrast agent in the present invention, the cell may be a stem cell.

The composition according to one embodiment of the present invention may comprise any one of conjugates above-mentioned.

According to another embodiment of the composition in the present invention, the active ingredient may be for treatment or prevention of disease, and the composition may be pharmaceutical composition.

According to another embodiment of the composition in the present invention, the active ingredient may be the active ingredient for functional cosmetics, and the composition may be cosmetic composition.

According to another embodiment of the composition in the present invention, the active ingredient may be the active ingredient for functional health food, and the composition may be health food composition.

The cytoplasm targeting delivery system of active ingredient according to the one embodiment of the present invention may comprise any one of conjugates mentioned above, wherein the carrier peptide moves into a cytoplasm locally and performs a role of local cytoplasm delivering the mentioned active ingredients, wherein the peptide having above 80% homology of amino acid sequence with above-mentioned sequence and the fragment of the same are the peptides that maintain cell penetrating ability of any one amino acid sequences of SEQ ID NO: 1 to SEQ ID NO: 156.

According to another embodiment of the method in the present invention, the method may be for delivering the active ingredient locally into mitochondria inside a cell.

According to another embodiment of the cell penetrating peptide in the present invention, the above-mentioned carrier peptide may be the peptide having any one or more amino acid sequences of SEQ ID NO: 1 to SEQ ID NO: 156.

The polynucleotide according to the present invention may encode above-mentioned cell penetrating peptide.

The vector according to the present invention may comprise above-mentioned polynucleotide.

The transformed cell according to the present invention may comprise above-mentioned vector.

### INDUSTRIAL APPLICABILITY

Active ingredients which are difficult to be transported inside a cell can be easily transported inside a cell by using the peptide, or the conjugate of the peptide and active ingredients, disclosed in the present invention. This means that efficacy of active ingredients can be increased and therefore the dosage can be lowered. As a result, side effects due to a drug administration can be minimized and effectiveness of treatment can be increased. Especially, as delivering drugs locally into mitochondria, mitochondria related diseases or disorders can be improved, and the effectiveness of prophylaxis and treatment of diseases can be increased. In a case of cosmetics, with a small amount of active ingredients, it can create an outstanding effect. By conjugating a peptide with a contrast substance, it can be used as a contrast substance to monitor a process of cell transplantation or transplanted cells in a cell treatment. Especially, it can be effectively used as a contrast substance for stem cells injected within a body.

### BRIEF DESCRIPTION OF THE DRAWINGS

FIG. 1 to FIG. 23 represents illustrating that the cellular uptake of the cell numbers from a HeLa cell treated with after the FITC fused to the peptide of SEQ: 1 to SEQ ID NO: 156, and analyzed by FACS. The control cells were treated only FITC.

FIG. 24 to FIG. 43 represents illustrating that the cellular uptake of the cell numbers from a Hur7 cell treated with after the FITC fused to the peptide of SEQ: 1 to SEQ ID NO: 156, and analyzed by FACS. The control cells were treated only FITC.

FIG. 44 to FIG. 58 represents illustrating that the cellular uptake of the cell numbers from a human T lymphocyte cell line (Jurket) treated with after the FITC fused to the peptide of SEQ: 1 to SEQ ID NO: 156, and analyzed by FACS. The control cells were treated only FITC.

FIG. 59 to FIG. 72 represents illustrating that the result of toxicity and cell viability from a HeLa cell treated with after the FITC fused to the peptide of SEQ: 1 to SEQ ID NO: 156, and analyzed by flow cytometry (Flow cytometry). The control cells were treated only FITC.

### DETAILED DESCRIPTION OF THE INVENTION

Proteins, Nucleic acids, Peptides or virus etc. have big potentials to be used as therapeutic substances. However, their uses are limited because they cannot penetrate tissues and cell membrane due to molecular level sizes. Although, the size of molecules is small, they cannot penetrate lipid-bilayer due to structure or characteristics of the molecules. Thus, through the use of electroporation, heat shock, etc., there were attempts to transport proteins, nucleic acids, peptides or viruses inside the cell; it was difficult to transfer those without neither damaging cell membrane nor keeping the active states of above molecules. There have been many studies conducted since TAT (Trans-Activating Transcriptional activator) protein derived from HIV (Human Immuno-deficiency Virus) has shown to work as cell penetrating peptide which can transport huge active substances inside the cell. Specifically, there have been studies conducted about substances that can transport huge molecules such as proteins, nucleic acids, peptides or virus inside the cell without causing any toxicity, unlike TAT protein which causes toxicity inside the cell. Therefore, the present invention was completed as present inventors have found that peptides derived from telomerase have outstanding efficacy as cell penetrating peptide without a noticeable toxicity.

Peptides are disclosed in SEQ ID NO: 1 to SEQ ID NO: 156 shown in Table 1 below. SEQ ID NO: 157 is a full length sequence of the Human telomerase protein. The “name” in Table 1 below was used for distinction of peptides. In a different specific embodiment of the present invention, more than one peptide of the mentioned peptides in SEQ ID NO: 1 to SEQ ID NO: 156 comprise a “synthetic peptide”, a synthesized peptide of selected areas of the telomerase. In the present specification, the term “pep” herein relates to a peptide that has any one amino sequence among SEQ ID NO: 1 to SEQ ID NO: 156, or a peptide comprising of amino acid sequence above 80% homology with above-mentioned sequences, or a peptide fragment of above-mentioned peptides.

TABLE 1

| SEQ ID | | Position in | | |
|---|---|---|---|---|
| NO | NAME | Telomerase | Sequence | Length |
| 1. | pep2 | [660-689] | ALFSVLNYERARRPGLLGASVLGLDDIHRA | 30 aa |
| 2. | pep3 | [663-677] | SVLNYERARRPGLLG | 15 aa |
| 3. | pep4 | [674-683] | GLLGASVLGL | 10 aa |
| 4. | pep5 | [615-624] | ALLTSRLRFI | 10 aa |
| 5. | pep6 | [613-621] | RPALLTSRL | 9 aa |
| 6. | pep7 | [653-661] | RLTSRVKAL | 9 aa |
| 7. | pep8 | [691-705] | RTFVLRVRAQDPPPE | 15 aa |
| 8. | pep9 | [653-667] | RLTSRVKALFSVLNY | 15 aa |
| 9. | pep10 | [651-665] | AERLTSRVKALFSVL | 15 aa |
| 10. | pep11 | [667-675] | YERARRPGL | 9 aa |
| 11. | pep12 | [675-683] | LLGASVLGL | 9 aa |
| 12. | pep13 | [680-689] | VLGLDDIHRA | 10 aa |
| 13. | pep14 | [677-686] | GASVLGLDDI | 10 aa |
| 14. | pep15 | [660-669] | ALFSVLNYER | 10 aa |
| 15. | pep16 | [663-672] | SVLNYERARR | 10 aa |
| 16. | pep17 | [679-688] | SVLGLDDIHR | 10 aa |
| 17. | pep18 | [662-671] | FSVLNYERAR | 10 aa |
| 18. | pep19 | [666-675] | NYERARRPGL | 10 aa |
| 19. | pep20 | [667-676] | YERARRPGLL | 10 aa |
| 20. | pep21 | [672-681] | RPGLLGASVL | 10 aa |
| 21. | pep22 | [668-676] | ERARRPGLL | 9 aa |
| 22. | pep23 | [680-688] | VLGLDDIHR | 9 aa |
| 23. | pep24 | [663-671] | SVLNYERAR | 9 aa |
| 24. | pep25 | [664-672] | VLNYERARR | 9 aa |
| 25. | pep26 | [670-678] | ARRPGLLGA | 9 aa |
| 26. | pep27 | [673-681] | PGLLGASVL | 9 aa |
| 27. | pep28 | [671-679] | RRPGLLGAS | 9 aa |
| 28. | pep29 | [660-668] | ALFSVLNYE | 9 aa |
| 29. | pep30 | [674-682] | GLLGASVLG | 9 aa |
| 30. | pep31 | [679-687] | SVLGLDDIH | 9 aa |
| 31. | pep32 | [668-675] | ERARRPGL | 8 aa |
| 32. | pep33 | [670-677] | ARRPGLLG | 8 aa |
| 33. | pep34 | [671-681] | GLLGASVL | 8 aa |
| 34. | pep35 | [669-676] | RARRPGLL | 8 aa |
| 35. | pep36 | [676-683] | LGASVLGL | 8 aa |
| 36. | pep37 | [563-577] | VTETTFQKNRLFFYR | 8 aa |
| 37. | pep38 | [573-587] | LFFYRKSVWSKLQSI | 15 aa |
| 38. | pep39 | [583-597] | KLQSIGIRQHLKRVQ | 15 aa |
| 39. | pep40 | [603-617] | EAEVRQHREARPALL | 15 aa |
| 40. | pep41 | [613-627] | RPALLTSRLRFIPKP | 15 aa |
| 41. | pep42 | [623-637] | FIPKPDGLRPIVNMD | 15 aa |
| 42. | pep43 | [643-657] | RTFRREKRAERLTSR | 15 aa |
| 43. | pep45 | [683-697] | LDDIHRAWRTFVLRV | 15 aa |
| 44. | pep46 | [693-707] | FVLRVRAQDPPPELY | 15 aa |
| 45. | pep47 | [721-735] | PQDRLTEVIASIIKP | 15 aa |
| 46. | pep48 | [578-592] | KSVWSKLQSIGIRQH | 15 aa |
| 47. | pep49 | [593-608] | LKRVQLRELSEAEVRQ | 16 aa |
| 48. | pep50 | [1-20] | MPRAPRCRAVRSLLRSHYRE | 20 aa |
| 49. | pep51 | [21-40] | VLPLATFVRRLGPQGWRLVQ | 20 aa |
| 50. | pep52 | [41-60] | RGDPAAFRALVAQCLVCVPW | 20 aa |
| 51. | pep53 | [61-80] | DARPPPAAPSFRQVSCLKEL | 20 aa |
| 52. | pep54 | [81-100] | VARVLQRLCERGAKNVLAFG | 20 aa |
| 53. | pep55 | [101-120] | FALLDGARGGPPEAFTTSVR | 20 aa |
| 54. | pep56 | [121-140] | SYLPNTVTDALRGSGAWGLL | 20 aa |
| 55. | pep57 | [141-160] | LRRVGDDVLVHLIARCALFV | 20 aa |
| 56. | pep58 | [161-180] | LVAPSCAYWVCGPPLYQLGA | 20 aa |
| 57. | pep59 | [181-200] | ATQARPPPHASGPRRRLGCE | 20 aa |
| 58. | pep60 | [201-220] | RAWNHSVREAGVPLGLPAPG | 20 aa |
| 59. | pep61 | [221-240] | ARRRGGSASRSLPLPKRPRR | 20 aa |
| 60. | pep62 | [241-260] | GAAPEPERTPVGQGSWAHPG | 20 aa |
| 61. | pep63 | [261-280] | RTRGPSDRGFCVVSPARPAE | 20 aa |
| 62. | pep64 | [280-300] | EATSLEGALSGTRHSHPSVG | 20 aa |
| 63. | pep65 | [301-320] | RQHHAGPPSTSRPPRPWDTP | 20 aa |
| 64. | pep66 | [321-340] | CPPVYAETKHFLYSSGDKEQ | 20 aa |
| 65. | pep67 | [341-350] | LRPSFLLSSLRPSLTGARRL | 20 aa |
| 66. | pep68 | [361-380] | VETIFLGSRPWMPGTPRRLP | 20 aa |
| 67. | pep69 | [381-400] | RLPQRYWQMRPLFLELLGNH | 20 aa |
| 68. | pep70 | [401-420] | AQCPYGVLLKTHCPLRAAVT | 20 aa |
| 69. | pep71 | [421-440] | PAAGVCAREKPQGSVAAPEE | 20 aa |
| 70. | pep72 | [441-460] | EDTDPRRLVQLLRQHSSPWQ | 20 aa |
| 71. | pep74 | [481-500] | RHNERRFLRNTKKFISLGKH | 20 aa |
| 72. | pep75 | [501-520] | AKLSLQELTWKMSVRDCAWL | 20 aa |
| 73. | pep76 | [521-540] | RRSPGVGCVPAAEHRLREEI | 20 aa |
| 74. | pep77 | [541-560] | LAKFLHWLMSVYVVEGGRSF | 20 aa |
| 75. | pep78 | [561-580] | FYVTETTFQKNRLFFYRKSV | 20 aa |
| 76. | pep79 | [581-600] | WSKLQSIGIRQHLKRVQLRE | 20 aa |
| 77. | pep80 | [601-620] | LSEAEVRQHREARPALLTSR | 20 aa |
| 78. | pep81 | [621-640] | LRFIPKPDGLRPIVNMDYVV | 20 aa |
| 79. | pep82 | [641-660] | GARTFRREKRAERLTSRVKA | 20 aa |
| 80. | pep83 | [661-680] | LFSVLNYERARRPGLLGASV | 20 aa |
| 81. | pep85 | [701-720] | DPPPELYFVKVDVTGAYDTI | 20 aa |
| 82. | pep86 | [721-740] | PQDRLTEVIASIIKPQNTYC | 20 aa |
| 83. | pep87 | [741-760] | VRRYAVVQKAAHGEVRKAFK | 20 aa |
| 84. | pep88 | [761-780] | SHVSTLTDLQPYMRQFVAHL | 20 aa |
| 85. | pep89 | [781-800] | QETSPLRDAVVIEQSSSLNE | 20 aa |
| 86. | pep90 | [801-820] | ASSGLFDVFLRFMCHHAVRI | 20 aa |
| 87. | pep91 | [821-840] | RGKSYVQCQGIPQGSILSTL | 20 aa |
| 88. | pep92 | [841-860] | LCSLCYGDMENKLFAGIRRD | 20 aa |
| 89. | pep93 | [861-880] | GLLLRLVDDFLLVTPHLTHA | 20 aa |
| 90. | pep94 | [881-900] | KTFLRTLVRGVPEYGCVVNL | 20 aa |
| 91. | pep95 | [901-920] | RKTVVNFPVEDEALGGTAFV | 20 aa |
| 92. | pep96 | [921-940] | QMPAHGLFPWCGLLLDTRTL | 20 aa |
| 93. | pep97 | [941-960] | EVQSDYSSYARTSIRASLTF | 20 aa |
| 94. | pep99 | [981-1000] | KCHSLFLDLQVNSLQTVCTN | 20 aa |
| 95. | pep100 | [1001-1020] | IYKILLLQAYRFHACVLQLP | 20 aa |
| 96. | pep101 | [1021-1040] | FHQQVWKNPTFFLRVISDTA | 20 aa |
| 97. | pep102 | [1041-1060] | SLCYSILKAKNAGMSLGAKG | 20 aa |
| 98. | pep103 | [1061-1080] | AAGPLPSEAVQWLCHQAFLL | 20 aa |
| 99. | pep104 | [1081-1100] | KLTRHRVTYVPLLGSLRTAQ | 20 aa |
| 100. | pep105 | [1101-1120] | TQLSRKLPGTTLTALEAAAN | 20 aa |
| 101. | pep106 | [1121-1132] | PALPSDFKTILD | 20 aa |
| 102. | pep107 | [1-10] | MPRAPRCRAV | 10 aa |
| 103. | pep108 | [11-30] | RSLLRSHYREVLPLATFVRR | 20 aa |
| 104. | pep109 | [31-50] | LGPQGWRLVQRGDPAAFRAL | 20 aa |
| 105. | pep110 | [51-70] | VAQCLVCVPWDARPPPAAPS | 20 aa |
| 106. | pep111 | [71-90] | FRQVSCLKELVARVLQRLCE | 20 aa |
| 107. | pep112 | [91-110] | RGAKNVLAFGFALLDGARGG | 20 aa |
| 108. | pep113 | [111-130] | PPEAFTTSVRSYLPNTVTDA | 20 aa |
| 109. | pep114 | [131-150] | LRGSGAWGLLLRRVGDDVLV | 20 aa |
| 110. | pep115 | [151-170] | HLLARVALFVLVAPSCAYQV | 20 aa |
| 111. | pep116 | [171-190] | CGPPLYQLGAATQARPPPHA | 20 aa |
| 112. | pep117 | [191-210] | SGPRRRLGCERAWNHSVREA | 20 aa |
| 113. | pep118 | [211-230] | GVPLGLPAPGARRRGGSASR | 20 aa |
| 114. | pep119 | [231-250] | SLPLPKRPRRGAAPEPERTP | 20 aa |
| 115. | pep120 | [251-270] | VGQGSWAHPGRTRGPSDRGF | 20 aa |
| 116. | pep121 | [271-290] | CVVSPARPAEEATSLEGALS | 20 aa |
| 117. | pep122 | [291-310] | GTRHSHPSVGRQHHAGPPST | 20 aa |
| 118. | pep123 | [311-330] | SRPPRPWDTPCPPVYAETKH | 20 aa |
| 119. | pep124 | [331-350] | FLYSSGDKEQLRPSFLLSSL | 20 aa |
| 120. | pep125 | [351-370] | RPSLTGARRLVETIFLGSRP | 20 aa |
| 121. | pep126 | [371-390] | WMPGTPRRLPRLPQRYWQMR | 20 aa |
| 122. | pep127 | [391-410] | PLFLELLGNHAQCPYGVLLK | 20 aa |
| 123. | pep128 | [411-430] | THCPLRAAVTPAAGVCAREK | 20 aa |
| 124. | pep129 | [431-450] | PQGSVAAPEEEDTDPRRLVQ | 20 aa |
| 125. | pep130 | [451-470] | LLRQHSSPWQVYGFVRACLR | 20 aa |
| 126. | pep131 | [471-490] | RLVPPGLWGSRHNERRFLRN | 20 aa |
| 127. | pep132 | [491-510] | TKKFISLGKHAKLSLQELTW | 20 aa |
| 128. | pep133 | [511-530] | KMSVRDCAWLRRSPGVGCVP | 20 aa |
| 129. | pep134 | [531-550] | AAEHRLREEILAKFLHWLMS | 20 aa |
| 130. | pep135 | [551-570] | VYVVELLRSFFYVTETTFQK | 20 aa |
| 131. | pep136 | [571-590] | NRLFFYRKSVWSKLQSIGIR | 20 aa |
| 132. | pep137 | [591-610] | QELKRVQLRELSEAEVRQHR | 20 aa |
| 133. | pep138 | [611-630] | EARPALLTSRLRFIPKPDGL | 20 aa |
| 134. | pep139 | [631-650] | RPIVNMDYVVGARTFRREKR | 20 aa |
| 135. | pep140 | [651-670] | AERLTSRVKALFSVLAYERA | 20 aa |
| 136. | pep141 | [671-690] | RRPGLLGASVLGLDDIHRAW | 20 aa |
| 137. | pep142 | [691-710] | RTFVLRVRAQDPPPELYFVK | 20 aa |
| 138. | pep143 | [711-730] | VDVTGAYDTIPQDRLTEVIA | 20 aa |
| 139. | pep145 | [751-770] | AHGHVRKAFKSHVSTLTDLQ | 20 aa |
| 140. | pep146 | [771-790] | PYMRQFVAHLQETSPLRDAV | 20 aa |
| 141. | pep147 | [791-810] | VIEQSSSLNEASSGLFDVFL | 20 aa |
| 142. | pep148 | [811-830] | RFMCHHAVRIRGKSYVQCQG | 20 aa |
| 143. | pep149 | [831-850] | IPQGSILSTLLCSLCYGDME | 20 aa |
| 144. | pep150 | [851-870] | NKLFAGIRRDGLLLRLVDDF | 20 aa |
| 145. | pep151 | [871-890] | LLVTPHLTHAKTFLRLTVRG | 20 aa |
| 146. | pep152 | [891-910] | VPEYGCVVNLRKTVVNFPVE | 20 aa |
| 147. | pep153 | [911-930] | DEALGGTAFVQMPAHGLFPW | 20 aa |
| 148. | pep154 | [931-950] | CGLLLDTRTLEVQSDYSSYA | 20 aa |
| 149. | pep156 | [971-990] | RRKLFGVLRLKCHSLFLDLQ | 20 aa |
| 150. | pep157 | [991-1010] | VNSLQTVCTNIYKILLLQAY | 20 aa |
| 151. | pep158 | [1011-1030] | RFHACVLQLPFHQQVWKNPT | 20 aa |
| 152. | pep159 | [1031-1050] | FFLRVISDTASLCYSILKAK | 20 aa |
| 153. | pep160 | [1051-1070] | NAGMSLGAKGAAGPLPSEAV | 20 aa |
| 154. | pep161 | [1071-1090] | QWLCHQAFLLKLTRHRVTYV | 20 aa |
| 155. | pep162 | [1091-1110] | PLLGSLRTAQTQLSRKLPGT | 20 aa |
| 156. | pep163 | [1111-1132] | TLTALEAAANPALPSDFKTILD | 20 aa |
| 157. | Telomerase | [1-1132] | MPRAPRCRAVRSLLRSHYREVLPLATFVRRLGPQGWRL | 1132 |
| | | | VQRGDPAAFRALVAQCLVCVPWDARPPPAAPSFRQVSC | |
| | | | LKELVARVLQRLCERGAKNVLAFGFALLDGARGGPPEA | |
| | | | FTTSVRSYLPNTVTDALRGSGAWGLLLRRVGDDVLVHL | |
| | | | LARCALFVLVAPSCAYQVCGPPLYQLGAATQARPPPHA | |
| | | | SGPRRRLGCERAWNHSVREAGVPLGLPAPGARRRGGSA | |
| | | | SRSLPLPKRPRRGAAPEPERTPVGQGSWAHPGRTRGPS | |
| | | | DRGFCVVSPARPAEEATSLEGALSGTRHSHPSVGRQHH | |
| | | | AGPPSTSRPPRPWDTPCPPVYAETKFLYSSGDKEQLRP | |
| | | | SFLLSSLRPSLTGARRLVETIFLGSRPWMPGTPRRLPR | |
| | | | LPQRYWQMRPLFLELLGNHAQCPYGVLLKTHCPLRAAV | |
| | | | TPAAGVCAREKPQGSVAAPEEEDTDPRRLVQLLRQHSS | |
| | | | PWQVYGFVRACLRRLVPPGLWGSRHNERRFLRNTKKFI | |
| | | | SLGKHAKLSLQELTWKMSVRDCAWLRRSPGVGCVPAAE | |
| | | | HRLREEILAKFLHWLMSVYVVELLRSFFYVTETTFQKN | |
| | | | RLFFYRKSVWSKLQSIGIRQELKRVQLRELSEAEVRQH | |
| | | | REARPALLTSRLRFIPKPDGLRPIVNMDYVVGARTFRR | |
| | | | EKRAERLTSRVKALFSVLNYERARRPGLLGASVLGLDD | |
| | | | IHRAWRTFVLRVRAQDPPPELYFVKVDVTGAYDTIPQD | |
| | | | RLTEVIASIIKPQNTYCVRRYAVVQKAAHGHVRKAFKS | |
| | | | HVSTLTDLQPYMRQFVAHLQETSPLRDAVVIEQSSSLN | |
| | | | EASSGLFDVFLRFMCHHAVRIRGKSYVQCQGIPQGSIL | |
| | | | STLLCSLCYGDMENKLFAGIRRDGLLLRLVDDFLLVTP | |
| | | | HLTHAKTFLRTLVRGVPEYGCVVNLRKTVVNFPVEDEA | |
| | | | LGGTAFVQMPAHGLFPWCGLLLDTRTLEVQSDYSSYAR | |
| | | | TSIRASLTFNRGFKAGRNMRRKLFGVLRLKCHSLFLDL | |
| | | | QVNSLQTVCTNIYKILLLQAYRFHACVLQLPFHQQVWK | |
| | | | NPTFFLRVISDTASLCYSILKAKNAGMSLGAKGAAGPL | |
| | | | PSEAVQWLCHQAFLLKLTRHRVTYVPLLGSLKRTAQTQ | |
| | | | LSRKLPGTTLTALEAAANPALPSDFKTILD | |
| 164 | pep1 | [611-626] | EARPALLTSRLRFIPK | 16 aa |

In one embodiment of the present invention the polynucleotide codes a peptide comprising at least one amino acid sequence of SEQ ID NO: 1 to SEQ ID NO: 156, a peptide having above 80% homology with above-mentioned sequences, or a peptide being a fragment of the above-mentioned peptides. The polynucleotide mentioned above enables production of the peptides in large quantities. For example, cultivation of vectors that include polynucleotides encoding peptides allows production of peptides in large quantities.

The peptides disclosed herein can include a peptide comprising amino acid sequence above 80%, above 85%, above 90%, above 95%, above 96%, above 97%, above 98%, or above 99% homology. Moreover, the peptides disclosed in the present invention can include a peptide comprising any one amino sequence among SEQ ID NO: 1 to SEQ ID NO: 156 or its fragments, and a peptide with more than 1 transformed amino acid, more than 2 transformed amino acid, more than 3 transformed amino acid, more than 4 transformed amino acid, more than 5 transformed amino acid, more than 6 transformed amino acid, or more than 7 transformed amino acid.

In one embodiment of the present invention, changes in amino acid sequence belong to the modification of peptide's physical and chemical characteristics. For example, amino acid transformation can be performed by improving thermal stability of the peptide, altering substrate specificity, and changing the optimal pH.

The term “amino acid” herein includes not only the 22 standard amino acids that are naturally integrated into peptide but also the D-isomers and transformed amino acids. Therefore, in a specific embodiment of the present invention, a peptide herein includes a peptide having D-amino acids. On the other hand, a peptide may include non-standard amino acids such as those that have been post-translationally modified. Examples of post-translational modification include phosphorylation, glycosylation, acylation (including acetylation, myristorylation, plamitoylation), alkylation, carboxylation, hydroxylation, glycation, biotinylation, ubiquitinylation, transformation in chemical properties (e.g. β-removing deimidation, deamidation) and structural transformation (e.g. formation of disulfide bridge). Also, changes of amino acids are included and the changes of amino acids occur due to chemical reaction during the combination process with crosslinkers for formation of a peptide conjugate.

A peptide disclosed herein may be a wild-type peptide that has been identified and isolated from natural sources. On the other hand, when compared to peptide fragments of any one amino sequence among SEQ ID NO: 1 to SEQ ID NO: 156, the peptides disclosed herein may be artificial mutants that comprise one or more substituted, deleted and/or inserted amino acids. Amino acid alteration in wild-type polypeptide—not only in artificial mutants—comprises conservative substitution of amino acids that do not influence protein folding and or activation. Examples of conservative substitution belong to the group consisting of basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). The amino acid substitutions that do not generally alter the specific activity are known in the art of the present invention. Most common occurred alteration are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Asp/Gly, and the opposite alterations. Another example of conservative substitutions are shown in the following table 2.

TABLE 2

| Original | Examples of | Preferable residue |
|---|---|---|
| amino acid | residue substitution | substitution |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | ala | Ala |
| Ser (S) | thr | Thr |
| Thr (T) | ser | Ser |
| Trp (W) | tyr; phe | Tyr |
| Tyr (Y) | trp; phe; thr; ser | Phe |
| Val (V) | ile; leu; met; phe; ala; norleucine | Leu |

The substantial transformation of the biological properties of peptides are performed by selecting significantly different substitution in the following efficacies: (a) the efficacy in maintaining the structure of the polypeptide backbone in the area of substitution, such as sheet or helical three-dimensional structures, (b) the efficacy in maintaining electrical charge or hydrophobicity of the molecule in the target area, or (c) the efficacy of maintaining the bulk of the side chain. Natural residues are divided into groups by general side chain properties as the following:
(1) hydrophobicity: Norleucine, met, ala, val, leu, ile;
(2) neutral hydrophilicity: cys, ser, thr;
(3) acidity: asp, glu;
(4) basicity: asn, gln, his, lys arg;
(5) residue that affects chain orientation: gly, pro; and
(6) aromaticity: trp, tyr, phe.

Non-conservative substitutions may be performed by exchanging a member of the above classes to different classes. Any cystein residues that are not related in maintaining the proper three-dimensional structure of the peptide can typically be substituted into serine, thus increasing the oxidative stability of the molecule and preventing improper crosslinkage. Conversely, improvement of stability can be achieved by adding cysteine bond(s) to the peptide.

Altered types of amino acids variants of peptides are those that antibody glycosylation pattern changed. The term “change” herein relates to deletion of at least one carbohydrate residues that are found in a peptide and/or addition of at least one glycosylated residues that do not exist within a peptide

Glycosylation in peptides are typically N-connected or O-connected. The term “N-connected” herein relates to that carbohydrate residues are attached to the side chain of asparagine residues. As tripeptide sequences, asparagine-X-serine and asparagine-X-threonine (where the X is any amino acid except proline) are the recognition sequence for attaching carbohydrate residue enzymatically to the side chain of asparagine. Therefore, with the presence of one of these tripeptide sequences in a polypeptide, the potential glycosylation sites are created. “O-connected glycosylation” means attaching one of sugar N-acetylgalactosamine, galactose, or xylose to hydroxyl amino acids. The hydroxyl amino acids are most typically serine or threonine, but 5-hydroxyproline or 5-hydroxylysine can be used.

Addition of glycosylation site to a peptide is conveniently performed by changing amino acid sequence to contain tripeptide sequence mentioned above (for N-linked glycosylation sites). These changes may be made by addition of at least one serine or threonine residues to the first antibody sequence, or by substitution with those residues (for O-linked glycosylation sites).

In one embodiment of the present invention, cell penetrating peptide comprising any one amino acid sequences of SEQ ID NO: 1 to SEQ ID NO: 156, a peptide having above 80% homology of amino acid sequence with above-mentioned sequences, or a fragment of the above-mentioned peptides, is provided.

In one embodiment of the present invention, a pharmaceutical composition comprising peptide as a drug delivery system to transport more than one active ingredient is provided, wherein the pepdite comprises any one amino acid sequence of SEQ ID NO: 1 to SEQ ID NO: 156, the peptide has above 80% homology with above-mentioned sequence, or the peptide is a fragment of above-mentioned peptide.

A peptide comprising any one amino acid sequence of SEQ ID NO: 1 to SEQ ID NO: 156, a fragment of above-mentioned peptide, or a peptide having above 80% homology with above-mentioned sequence, is safe and has outstanding efficacy as cell penetrating peptide. Therefore, the peptide can be conjugated with a drug to transport the drug inside the cell.

In one embodiment of the present invention, a conjugate of a peptide and an active ingredient to be transported is provided, wherein the peptide comprises any one amino acid sequence of SEQ ID NO: 1 to SEQ ID NO: 156, the peptide is a fragment of above-mentioned peptide, or the peptide has above 80% homology with above-mentioned peptide. In one embodiment of the present invention, an active ingredient may be at least one selected from proteins, nucleic acids, peptides, lipids, glycolipids, minerals, sugars, contrast substances, drugs and chemical compounds. In one embodiment of the present invention, the active ingredients may be peptides. In one embodiment of the present invention, the active ingredients may be cytokines, antibody, antibody fragments, therapeutic enzymes, soluble receptors, or ligands.

A cell penetrating peptide disclosed herein means a peptide which can transport cargo from in vitro and/or in vivo to inside the cell. A “cargo” disclosed herein comprises all the substances that can be transported inside the cell via conjugation with a cell penetrating peptide, For example, all the substances which want to increase cell penetrating efficacy, specifically drugs, cosmetics, or active ingredients of health food, more specifically substances which cannot be transported inside the cell via general route, more specifically, sugars, nano-particles, biological formulation, viruses, contrast substances or other chemical compounds which can have proteins, nucleic acids, peptide, minerals, glucose as an example, but not limited to those. A “drug” disclosed herein is a broad concept including a substance to be transported for alleviation, prophylaxis, treatment or diagnosis of diseases, wounds, or specific symptom.

A “carrier peptide” disclosed herein is a peptide which can transport active ingredients to a targeted site via conjugation with active ingredients.

In one embodiment of the present invention, protein or peptide as a cargo comprises one or more of hormone, hormone analogue, enzyme, enzyme inhibitors, signal transfer proteins (or peptides), antibody and vaccine, but not limited to those. In one embodiment of the present invention, a nucleic acid is a molecule that can be spontaneous or artificial DNA or RNA molecules, either single-stranded or double-stranded. The nucleic acid molecule can be one or more nucleic acids of same type (for example, having a same nucleotide sequence) or nucleic acids of different types. The nucleic acid molecules comprise one or more DNA, cDNA, decoy DNA, RNA, siRNA, miRNA shRNA, stRNA, snoRNA, snRNA PNA, antisense oligomer, plasmid and other modified nucleic acids, but not limited to those. In one embodiment of the present invention, virus comprises the whole virus or the core of virus which includes nucleic acids of the virus. In one embodiment of the present invention, a chemical substance is a broad indication comprising a natural or synthetic substance which can act as a drug.

In one embodiment of the present invention, drug to be delivered into cells by a cell-permeable peptide is iposomes, micelles, nanoparticles, or quantum dots as drug delivery.

The term “contrast substance” disclosed herein is a broad indication comprising all the substances used to contrast structures or fluids within the body in medical imaging. An appropriate contrast substance comprises radiopaque contrast agent, paramagnetic contrast agent, superparamagnetic contrast agent, CT (computed tomography) and other contrast substances, but not limited to those. For example, a radiopaque contrast agent (for x-ray imaging) will comprise inorganic iodine compound and organic iodine compound (for example, diatrizoate), radiopaque metals and their salts (for example, silver, gold, platinum, etc.) and other radiopaque compounds (for example, calcium salts, barium salt such as barium sulfate, tantalum and oxidized tantalum). An appropriate paramagnetic contrast substance (for MR imaging) comprises gadolinium diethylene triaminepentaacetic acid (Gd-DTPA) and its derivatives, other gadolinium, manganese, iron, dysprosium, copper, europium, erbium, chrome, nickel and cobalt complex, for example, 1,4,7,10-tetraazacyclododecan-N,N′,N″,N′″-tetraacetic acid (DOTA), ethylenediaminetetraacetic acid (EDTA), 1,4,7,10-tetraazacyclododecan-N,-N′,N″-triacetic acid (DO3A), 1,4,7-triazacyclononane-N,N′,N″-TRIACETIC ACID (NOTA), 1,4,8,10-tetraazacyclotetradecane-N,N′,N″,N′″-tetraacetic acid (TETA), hydroxybenzylethylene-diamine diacetic acid (HBED). An appropriate superparamagnetic contrast substance (for MR imaging) comprises magnetite, super-paramagnetic iron oxide (SPIO), ultrasmall superparamagnetic iron oxide (USPIO) and monocrystailine iron oxide. Other appropriate contrast substances are iodinated, non-iodinated, ionic and non-ionic CT contrast agents, a contrast substance like spin-label or diagnostically effective agent.

Other examples of contrast substances comprise β-galactosidase, Green Fluorescent Protein, Cyan Fluorescent Protein, luciferase, but not limited to those, and a marker gene which codes for protein which can be easily detected when expressed within cells. Various labels such as radionuclide, flour, enzyme, enzyme-substrate, enzyme cofactor, enzyme inhibitor, ligands (especially hapten) can be used.

In one example of the present invention, a contrast substance is ferrocenecarboxylic acid of the below chemical formula 2. The structure of ferrocene is shown in the chemical formula 1.

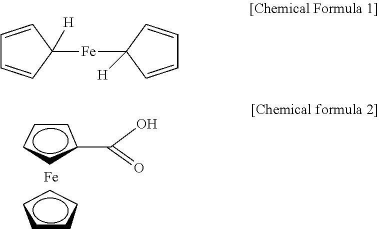

In one example of the present invention, a conjugate of cell penetrating peptide and a contrast substance is Ferrocenecarboxylic-pep (Ferrocenecarboxylic-pep) shown in the below chemical formula 3.

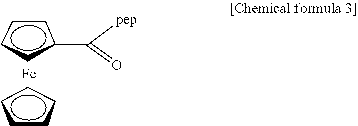

In one embodiment of the present invention, a peptide or composition can be fused with one or more detectable labels. Labels may be compounds which can be detected in chemical, physical or enzymatic responses, or compounds which generate signals directly or indirectly in the responses. Labeling and detecting after then can be performed according to the known method in the art (For example, Sambrook, J., and Russel, D. W.(2001); and Lottspeich, F., and Zorbas H. (1998) Bioanalytik, Spektrum Akademischer Verlag, Heidelberg/Berlin, Germany). Labels comprise fluorescent label, enzyme label, chromogenic label, luminescence label, radiation label, hapten, biotin, metal complex, metal and colloidal gold, but not limited to those. All forms of these labels are well known in this field of work, they can be commercially obtained from various suppliers.

In one embodiment of the present invention, a cargo can be directly combined with the peptide. In another embodiment of the present invention, a cargo can be combined to the peptide via various types of bonds such as covalent or non-covalent bonds. A cargo, for example, can be combined to the N-terminal or C-terminal of the peptide in one embodiment of the present invention. For example, a cargo can be bonded to the peptide by disulfide bonds or covalent bonds. The covalent bonds are the bonds that a cargo can be bonded to α-amine of N-terminal glutamate, or amine of C-terminal Lysine residues. Also, a peptide and a cargo can be combined via a non-covalent bond, which can have either a peptide or a cargo can encapsulate the other as a capsule form.

In another embodiment of the present invention, a peptide can be combined with a cargo via a linker. For example, a peptide can be combined with a cargo by binding a cargo to a linker after introducing a linker such as Hynic (6-hydrazinopyridine-3-carboxylic acid) linker, to the α-amine of N-terminal glutamate, or amine of C-terminal Lysine residues.

In another embodiment of the present invention, when a cargo is DNA or RNA, SH group (thiol group) is introduced to the peptide, and maleimide group is introduced to DNA or RNA, then, SH group of the peptide and maleimide group of DNA or RNA are combined, thus creating a bond between the cargo and the peptide.

In another embodiment of the present invention, when a cargo is a peptide or protein, DNA which expresses a cargo is combined with DNA which expresses a carrier peptide, and by expressing this, a cargo and a peptide can be combined as a form of fusion protein. Specific examples of combination by a fusion protein are as follows: when manufacturing primer for production of fusion protein, a nucleotide coding a carrier peptide is attached in front of a nucleotide expressing a cargo, and the obtained nucleotide is inserted to a vector such as pET vector using a restriction enzyme, and the nucleotide is expressed by transformation into a cell such as BL-21(DE3). At this time, a fusion protein is to be effectively expressed by treating it with an expression inducing agent like IPTG (isopropyl-1-thio-β-D-galactopyranoside). Then, the expressed fusion protein is purified by His tag purification, and is dialyzed with PBS, and is added to a kit to be concentrated by centrifugation under such condition for 5 to 20 mins at 2,000 to 4,000 rpm.

In one embodiment of the present invention, a carrier peptide is combined with dying substances, fluorescent substances, specifically FITC (fluorescein isothiocyanate) or GFP (Green Fluorescent Protein). In one embodiment of the present invention, FITC is combined with amino group (NH³⁺) of lysine at N-terminal or C-terminal of a carrier peptide. In the case of a peptide, where lysine does not exist at its terminal, the peptide can be combined with FITC via a linker including Lysine.

The carrier peptide disclosed herein which is the peptide comprising any one amino acid sequence of SEQ ID NO: 1 to SEQ ID NO: 156, or the peptide having above 80% homology of amino acid sequence with above-mentioned peptides, or a fragment of above-mentioned peptide, can be combined with a cargo at a mole fraction of 1:1, but it can be combined at mole fraction other than 1:1. For example, a mole fraction of CPP and a cargo may be more than 2:1, specifically, more than 2:1, more than 3:1, more than 4:1, more than 5:1, more than 6:1, more than 7:1, more than 8:1, more than 9:1 or more than 10:1. This means that numerous carrier peptide molecules can be combined with a cargo molecule. The numerous carrier peptide molecules can be combined in series or in parallel. “Combined in series” means that a carrier peptide and a cargo molecule are to be combined at terminal amino acids. “Combined in parallel” means that they are to be combined at a site other than terminal amino acids. On the other hand, the mole fraction of a carrier peptide and a cargo may be more than 1:2. This means that a carrier peptide molecule can be combined with numerous number of a cargo molecule. For example, a mole fraction of a carrier peptide and a cargo may be 1:2, specifically, more than 1:2, more than 1:3, more than 1:4, more than 1:5, more than 1:6, more than 1:7, more than 1:8, more than 1:9 or more than 1:10.

A movement pathway of the peptide combined with Fluorescein isothiocyanate can be easily found. Therefore, a carrier peptide in one embodiment of the present invention is to be used for cell imaging or detecting a pathway of drug delivery inside a cell.

In one embodiment of the present invention, a use of the peptide as a drug delivery carrier to transport more than one active ingredient is provided, wherein the peptide comprises any one amino acid sequence of SEQ ID NO: 1 to SEQ ID NO: 156, or the peptide is a fragment of above-mentioned peptide, or the peptide has above 80% homology of amino acid sequence with above-mentioned peptide. The use may indicate therapeutic or non-therapeutic use.

In one embodiment of the present invention, a method of delivering drugs inside a cell of a subject comprising a step of administering a composition comprising a drug; and the peptide is provided; wherein the peptide comprises any one amino acid sequence of SEQ ID NO: 1 to SEQ ID NO: 156, or the peptide is a fragment of above-mentioned peptide, or the peptide has above 80% homology of amino acid sequence with above-mentioned peptide,

In one embodiment of the present invention, a method of detecting drug delivery pathway comprising a step of applying the peptide and a contrast substance to a subject is provided; wherein the peptide comprises any one amino acid sequence of SEQ ID NO: 1 to SEQ ID NO: 156, or the peptide is a fragment of above-mentioned peptide, or the peptide has above 80% homology of amino acid sequence with above-mentioned peptide.

In one embodiment of the present invention, a method of detecting drug delivery pathway comprising a step of applying of the conjugate of the peptide and a contrast substance to a subject is provided; wherein the peptide comprises any one amino acid sequence of SEQ ID NO: 1 to SEQ ID NO: 156, or the peptide is a fragment of above-mentioned peptide, or the peptide has above 80% homology of amino acid sequence with above-mentioned peptide.

In one embodiment of the present invention, a kit for drug delivery into a cell of a subject containing the composition and an instruction is provided, wherein the composition comprises a conjugate of a peptide of the invention and a drug for delivery, wherein the peptide comprises any one amino acid sequence of SEQ ID NO: 1 to SEQ ID NO: 156 or the peptide is a fragment of above-mentioned peptide, or the peptide has above 80% homology of amino acid sequence with above-mentioned peptide, wherein the instruction includes at least one of administration dose, administration route, administration frequency, and indication of the composition.

In one embodiment of the present invention, cosmetic or food composition comprising an active ingredient; and the peptide is provided; wherein the peptide comprises amino acid sequence of SEQ ID NO: 1 to SEQ ID NO: 156, the peptide has above 80% homology of amino acid sequence with above-mentioned sequence, or the peptide is a fragment of the above-mentioned peptides. In another embodiment of the present invention, cosmetic or food composition comprising a conjugate of the peptide and active ingredients is provided; wherein the peptide comprises any one amino acid sequence of SEQ ID NO: 1 to SEQ ID NO: 156, the peptide has above 80% homology of amino acid sequence with above-mentioned sequence, or the peptide is a fragment of the above-mentioned peptides.

In one embodiment of the present invention, pharmaceutical, cosmetic or food composition with an outstanding ability to transport active ingredients inside a cell, comprising a conjugate of the peptide and an active ingredient, is provided; wherein the peptide comprises any one amino acid sequence of SEQ ID NO: 1 to SEQ ID NO: 156, the peptide has above 80% homology of amino acid sequence with above-mentioned sequence, or the peptide is a fragment of the above-mentioned peptides.

Mitochondria, as a central organelle in energy metabolism of a eukaryotic cell, is a first known intracellular organelle to be related to human diseases (Luft R, Ikkos D, Palmieri G. Ernster L, Afzelius B: A case of severe hypermetabolism of non thyroid origin with a defect in the maintenance of mitochondrial respiratory control: a correlated clinical, biochemical, and morphological study, J Clin Invest 41: 1776-804, 1962).

Since the mitochondria play an important role in control of energy metabolism of cell and apoptosis, they act as a major target for various therapeutic drugs. Also, this organelle is involved in control of the calcium concentration inside the cell, the mitochondrial respiratory chain acts as an electron transport system which is important in energy production, and it causes production of reactive oxygen species. As a result, the abnormal mitochondrial function has a close relationship with adult diseases such as diabetes, cardiomyopathy, infertility, blindness, renal/liver diseases, and stroke (Modica-Napolitano K S, Singh K K: April mitochondria as targets for detection and treatment of cancer. Expert Rev Mol Med 11:1-19, 2002). Also, it is being suggested that Mitochondrial genetic mutations to be involved in the outbreak of aging, degenerative neuronal disease and cancer etc.

The mitochondria targeting delivery system can be provided according to the one embodiment of the present invention may comprise any one of conjugates mentioned above, wherein the carrier peptide moves into intracellular mitochondria locally and performs a role of local intracellular mitochondria delivering the mentioned active ingredients, wherein the peptide haying above 80% homology of amino acid sequence with above-mentioned sequence and the fragment of the same are the peptides that maintain mitochondria targeting delivery system, the above-mentioned mitochondria targeting peptide may be the peptide having any one amino acid sequences of SEQ ID NO: 1 to SEQ ID NO: 156.

The mitochondria activity adjusting composition can be provided, wherein the composition comprises a conjugate of a peptide of the invention and a carrired peptid for delivery, wherein the carrier peptide moves into intracellular mitochondria locally and performs a role of local intracellular mitochondria delivering the mentioned active ingredients, wherein the peptide having above 80% homology of amino acid sequence with above-mentioned sequence and the fragment of the same are the peptides that maintain mitochondria targeting delivery system, the above-mentioned mitochondria targeting peptide may be the composition having any one amino acid sequences of SEQ ID NO: 1 to SEQ ID NO: 156.

The mitochondria activity adjusting composition according to the one embodiment of the present invention, wherein the composition for the treatment of a mitochondrial related diseases or disorder, prevention, inhibitory of progress, or relief of symptoms as an a pharmaceutical composition, wherein the active ingredient will be treated for a mitochondrial related diseases or disorder, prevention, inhibitory of progress, or relief of symptoms.

The “Mitochondrial related diseases” disclosed herein comprise Huntington's disease, amyotriophic lateral sclerosis, MELAS (Mitochondrial Encephalomyopathy with Lactic Acidemia and Stroke-like episodes); MERRF (Myoclonus, epilepsy, and myopathy with ragged red fibers; NARP/MILS (Neurogenic muscular weakness, ataxia, retinitis pigmentosa/Maternally inherited leigh syndrome); LHON (Lebers hereditary optic neuropathy); KSS (Kearns-Sayre Syndrome); PMPS (Pearson Marrow-Pancreas Syndrome); CPEO (Chronic progressive external opthalnoplegia); Reye's syndrome; Alper's syndrome; Multiple mtDNA deletion syndrome; mtDNA depletion syndrome; Complex I deficiency; Complex II (SDH) deficiency; Complex III deficiency; Cytochrome c oxidase (COX, Complex IV) deficiency; Complex V deficiency; Adenine nucleotide translocator (ANT) deficiency; Pyruvate dehydrogenase (PDH) deficiency; Ethyl malonic acid aciduria having lactic acid acidemia; 3-methyl glutaconic acid aciduria having lactic acid acidemia; refractoriness epilepsy representing a decline during infection; Asperger's syndrome representing a decline during infection; Autism representing a decline during infection; Attention deficit hyperactivity disorder(ADHD); Cerebral palsy representing a decline during infection; Alexia representing a decline during infection; Maternal hereditary thrombocytopenia; Leukemia; MNGIE (Mitochondrial myopathy, peripheral and autonomic neuropathy, gastrointestinal dysfunction, and epilepsy); MARIAHS syndrome (Mitochondrial ataxia, recrudescent infection, aphasia, hypouricemia/hypomyelination, seizure and dicarboxylic acid aciduria); ND6 dystonia; Cyclic vomiting syndrome representing a decline during infection; 3-hydroxyisobutyric acid aciduria having lactic acid acidemia; Diabetes having lactic acid acidemia; Uridine reactive neural syndrome (URNS); Familial bilateral striatum necrosis (FBSN); Hearing loss related with aminoglycoside; Relaxed myocardiopathy; Spleen lymphoma; Wolframs syndrome; Multiple mitochondria DNA deletions syndrome; and Renal tubular acidosis/diabetes/ataxia syndrome, but not limited to those.

In another embodiment of the present invention, nucleic acid molecules encoding above-mentioned polypeptides are provided. The nucleic acid molecules, for example, have base sequences of GAA GCG CGC CCG GCG CTG CTG ACC AGC CGC CTG CGC TTT ATT CCG AAA (SEQUENCE NO: 181). The nucleic acids can be introduced into the host cell according to a known method to those skilled in the art. For example, the known methods may be transformation method by calcium phosphate method, liposome, electroporation, contacting a virus and a cell, or micro injection directly into the cell, etc. The host cell is higher eukaryotic cell, for example, mammalian cells, or lower eukaryotic cells, such as a yeast cell, or prokaryotic cells, such as a bacterial cell. The prokaryotic host cells appropriate for transformation may be the species which belong to E. coli, Bacillus subtillis, Salmonella typhimurium, Pseudomonas, Streptomyces, and Micro bacteria species, as examples.

The vector including above-mentioned nucleic acid molecules is generally recombinant expression vector and it comprises, origin of replication enabling a host cell transformation, and a selectable marker (for example, dihydrofolate reductase for eukaryotic cell culture, or tolerance of neomycin, tolerance of tetra-cycline or ampicillin in E.coli, or S. cerevisiae TRP1 gene), and the promoter for controlling transcription of protein coating sequences. Available expression vectors are, for example, known bacterial plasmids such as SV40, derivatives of pcDNA, and known bacterial plasmids such as colE1, pCR1, pBR322, pMal-C2, pET, pGEX (Smith, et al., Gene 67:31-40(1988)), plasmids such as pMB9 and its derivative RP4, phage DNA which is the same as numerous derivatives of phage I such as NM989, phage DNA such as M13 and single-stranded phage DNA of filament type; yeast plasmid, for example, phage DNA or vector induced from a combination of modified plasmid for using expression suppression sequences and phage DNA. The mammalian expression vectors comprise origin of replication, an appropriate promoter and an enhancer. Also, they can comprise compulsory ribosome binding sites, polyadenylation sites, splice donor, and receptor part, transcription termination sequences, and 5′ planking non-transcriptional sequences. The mammalian expression vectors can comprise an inducible promoter, for example, a vector containing dihydrofolate reductase promoter, any expression vectors containing DHFR expression cassette or DHFR/methotrexate co-amplification vector such as pED.(Randal J, kaufman, 1991. Randal J. Kaufman, Current Protocols in Molycular Biology, 16, 12(1991)). Or, glutamine synthetase/methionine sulfoximine co-amplification vector, for example, pEE14 (Celltech), Epstein-Barr-Virus (EBV), or a vector directing episomal expression under the control of nuclear antigen (EBNA), for example, pREP4 (Invitrogen), pCEP4 (Invitrogen), pMEP4 (Invitrogen), pREP8 (Invitrogen), pREP9 (Invitrogen) and pEBVHis (Invitrogen) can be used. Selectable mammalian expression vectors are Rc/CMV (Invitrogen) and pRc/RSV (Invitrogen) etc. Vaccinia virus mammalian expression vectors which can be used in the present invention are pSC11, pMJ601, pTKgptF1S, etc.

Yeast expression vector system to be used in the present invention is non-fusion pYES2 vector (Invitrogen), fusion pYESHisA, B, C (Invitrogen), pRS vector, etc.

The above-mentioned vectors can be introduced to various cells, such as mammalian cells which is especially the human derived cells, or bacteria, yeast, fungi, insects, nematodes, and plant cells. The examples of appropriate cells are VERO cell, HELA cell, for example, ATCC No. CCL2, CHO cell line, for example, ATCC No. CCL61, COS cell, for example COS-7 cell and ATCC No. CRL 1650 cell, W138, BHK, HepG2, 3T3, for example, ATCC No. CRL6361, A549, PC12, K562 cell, 293 cell, Sf9 cell, for example, ATCC No. CRL1711 and Cv1 cell, such as ATCC No. CCL70, etc.

Other appropriate cells to be used in the present invention are prokaryotic host cell strain, for example, the strains belonging to E. coli (e.g. DH5-α strain), Bacillus subtilis, Salmonella typhimurium, Pseudomonas, Streptomyces and Staphylococcus.

In one embodiment of the present invention, the composition may contain 0.1 μg/mg to 1 mg/mg, specifically 1 μg/mg to 0.5 mg/mg, more specifically 10 μg/mg to 0.1 mg/mg of a peptide comprising any one amino acid sequence of SEQ ID NO: 1 to SEQ ID NO: 156, a peptide comprising amino acid sequence above 80% homology with above-mentioned sequence, or a fragment of above-mentioned peptide. When the peptide is contained in the above-mentioned range, all the safety and stability of the composition can be satisfied and appropriate in terms of cost-effectiveness.

In one embodiment of the present invention, the composition may have application with all animals including human, dog, chicken, pig, cow, sheep, guinea pig, and monkey.

In one embodiment of the present invention, the pharmaceutical composition may be administered through oral, rectal, transdermal, intravenous, intramuscular, intraperitoneal, in the bone marrow, epidural or subcutaneous means.

Forms of oral administration may be, but not limited to, tablets, pills, soft or hard capsules, granules, powders, solution, or emulsion. Forms of non-oral administration can be, but not limited to, injections, drips, lotions, ointments, gels, creams, suspensions, emulsions, suppository, patch, or spray.

In one embodiment of the present invention, the pharmaceutical composition, if necessary, may contain additives, such as diluents, excipients, lubricants, binders, disintegrants, buffers, dispersants, surfactants, coloring agents, aromatics or sweeteners. In one embodiment of the present invention, the pharmaceutical composition may be manufactured by conventional methods of the industry in the art.

In one embodiment of the present invention, the active ingredient of the medical composition may vary according to the patient's age, sex, weight, pathology and state, administration route, or prescriber's judgment. Dosage based on these factors is determined within levels of those skilled in the art, and the daily dose for example may be, but not limited to, 0.1 μg/kg/day to 1 g/kg /day, specifically 1 μg/kg/day to 10 mg/kg/day, more specifically the 10 μg/kg/day to 1 mg/kg/day, more specifically the 50 μg/kg/day to 100 μg/kg/day. In one embodiment of the present invention, the pharmaceutical composition may be administered, but not limited to, 1 to 3 times a day.

In one embodiment of the present invention, cosmetic composition may be provided in all forms appropriate for topical applications. For example, forms may be provided as solutions, emulsions obtained by dispersion of oil phase in water, emulsion obtained by dispersion of water in oil phase, suspension, solid, gel, powder, paste, foam or aerosol. These forms may be manufactured by conventional methods of the industry in the art.

In one embodiment of the present invention, the cosmetic composition may include, within levels that won't harm the main effect, other ingredients that may desirably increase the main effect. In one embodiment of the present invention, the cosmetic composition may additionally include moisturizer, emollient agents, surfactants, UV absorbers, preservatives, fungicides, antioxidants, pH adjusting agent, organic or inorganic pigments, aromatics, cooling agent or antiperspirant. The formulation ratio of the above-mentioned ingredients may be decided by those skilled in the art within levels that won't harm the purpose and the effects of the present invention, and the formulation ratio based on total weight of the cosmetic composition may be 0.01 to 5% by weight, specifically 0.01 to 3% by weight.

In one embodiment of the present invention, food composition is not limited to forms, but for example may be granules, powder, liquid, and solid forms. Each form may be formed with ingredients commonly used in the industry appropriately chosen by those skilled in the art, in addition to the active ingredient, and may increase the effect with other ingredients.

Decision for dosage on the above-mentioned active ingredient is within the level of those skilled in the art, and daily dosage for example may be 1 μg/kg/day to 10 mg/kg/day, more specifically the 10 μg/kg/day to 1 mg/kg/day, more specifically the 50 μg/kg/day to 100 μg/kg/day, but not limited to these numbers and can vary according to age, health status, complications and other various factors.

The terms used herein is intended to be used to describe the embodiments, not to limit the present invention. Terms without numbers in front are not to limit the quantity but to show that there may be more than one thing of the term used. The term “including”, “having”, “consisting”, and “comprising” shall be interpreted openly (i.e. “including but not limited to”).

Mention of range of numbers is used instead of stating separate numbers within the range, so unless it is explicitly stated, each number can be read as separate numbers integrated herein. The end values of all ranges are included in the range and can be combined independently.

Unless otherwise noted or clearly contradicting in context, all methods mentioned herein can be performed in the proper order. The use of any one embodiment and all embodiment, or exemplary language (e.g., that use “like ˜”), unless included in the claims, is used to more clearly describe the present invention, not to limit the scope of the present invention. Any language herein outside of the claims should not be interpreted as a necessity of the present invention. Unless defined otherwise, technical and scientific terms used herein have meaning normally understood by a person skilled in the art that the present invention belongs to.

The preferred embodiments of the present invention are the best mode known to the inventors to perform the present invention. It can become clear to those skilled in the art after reading the statements ahead of the variations in the preferred embodiments. The present inventors hope that those skilled in the art can use the variations adequately and present invention be conducted in other ways than listed herein. Thus, the present invention, as allowed by the patent law, includes equivalents, and variations thereof, of the key points of the invention stated in the appended claims. In addition, all possible variations within any combination of the above-mentioned components are included in the present invention, unless explicitly stated otherwise or contradicting in context. Although the present invention is described and shown by exemplary embodiments, those skilled in the art will understand well that there can be various changes in the form and details without departing from the spirit of the invention and range, defined by the claims below.

### Example 1: Synthesis of Peptide

The peptides of SEQ ID NO: 1 to SEQ ID NO: 156 were synthesized according to the existing method of solid phase peptide synthesis. In detail, the peptides were synthesized by coupling each amino acid from C-terminus through Fmoc solid phase peptide synthesis, SPPS, using ASP48S (Peptron, Inc., Daejeon ROK). Those peptides with their first amino acid at the C-terminus being attached to resin were used as follows:
NH₂-Lys(Boc)-2-chloro-Trityl Resin
NH₂-Ala-2-chloro-Trityl Resin
NH₂-Arg(Pbf)-2-chloro-Trityl Resin

All the amino acid materials to synthesize the peptide were protected by Fmoc at the N-terminus, and the amino acid residues were protected by Trt, Boc, t-Bu (t-butylester), Pbf (2,2,4,6,7-pentamethyl dihydro-benzofuran-5-sulfonyl) that can be dissolved in acid. Such as:
Fmoc-Ala-OH, Fmoc-Arg(Pbf)-OH, Fmoc-Glu(OtBu)-OH, Fmoc-Pro-OH, Fmoc-Leu-OH, Fmoc-Ile-OH, Fmoc-Phe-OH, Fmoc-Ser(tBu)-OH, Fmoc-Thr(tBu)-OH, Fmoc-Lys(Boc)-OH, Fmoc-Gln(Trt)-OH, Fmoc-Trp(Boc)-OH, Fmoc-Met-OH, Fmoc-Asn(Trt)-OH, Fmoc-Tyr(tBu)-OH, Fmoc-Ahx-OH, Trt-Mercaptoacetic acid.

HBTU[2-(1H-Benzotriazole-1-yl)-1,1,3,3-tetamethylaminium hexafluorophosphate]/HOBt [N-Hydroxybenzotriazole]/NMM [4-Methylmorpholine] were used as the coupling reagents. In 20% of DMF, piperidine was used to remove Fmoc. In order to remove the protection from residue or to separate the synthesized peptide from Resin, cleavage cocktail [TFA (trifluoroacetic acid)/TIS (triisopropylsilane)/EDT (ethanedithiol)/H₂O=92.5/2.5/2.5/2.5] was used.

Peptides were synthesized by using the solid phase scaffold by adding each amino acid with the sequential processes as follow; amino acid protection, coupling reaction, washing, and deprotection. After cutting off the synthecized peptide from the resin, it was purified by HPLC and verify for synthesis by MS, and then freeze-dried.

Specific peptide synthesis process is described by the following with example of Pep1 of SEQ ID NO: 164(EARPALLTSRLRFIPK).
1) Coupling

The amino acid (8 equivalent) protected with NH₂-Lys(Boc)-2-chloro-Trityl Resin was melted in coupling agent HBTU (8 equiv.)/HOBt (8 equiv.)/NMM (16 equiv.), and upon addition of DMF, the reaction mixture was incubated at room temperature for 2 hours, then washed sequentially with DMF, MeOH, and DMF.
2) Fmoc deprotection

Following the addition of 20% piperidine in DMF, the reaction mixture was incubated at room temperature for 5 minutes 2 times, then washed sequentially with DMF, MeOH, and DMF.
3) Make the basic framework of peptide by repeating reactions 1 and 2 repeatedly.
4) Cleavage: Add Cleavage Cocktail to the completely synthesized peptide and separate the peptide from the resin.
5) Add pre-chilled diethyl ether into the mixture, and then centrifuge the reaction mixture to precipitate out the peptides.
6) After purification by Prep-HPLC, check the molecular weight by LC/MS and lyophilize to obtain the peptides in a powder form.

### Example 2: Preparation of pep(CPP)-FITC Conjugate

### (1) Preparation of FITC-CPP Conjugate

A conjugate of the peptides with SEQ ID NO: 1 to SEQ ID NO: 156 combined with FITC was manufactured as follows, for example, a conjugate of pep1 (SEQ ID NO: 157) and FITC, in other words, FITC-linker-pep1 was manufactured as follows.

The basic framework of peptide, NH₂-linker-E(OtBu)-A-R(Pbf)-P-A-L-L-T(tbu) -S(tBu)-R(Pbf)L-R(Pbf)-F-I-P-K(Boc)-2-chloro-Trityl Resin) which was obtained. according to the manufacturing methods described in Example 1, was reacted with FITC. Specifically, FITC (fluorescein-5isothiocyanate) (8 equivalent) and DIPEA (N,N-Diisopropylethylamine) (16 equivalent) were melted in DMF. The DMF solution was added, and reacted at room temperature for 2 hours, then washed sequentially with DMF, MeOH and DMF. As a result, FITC-linker-E(OtBu)-A-R(Pbf)-P-A-L-L-T(tBu)-S(tBu) -R(Pbf)L-R(Pbf)-F-I-P-K(Boc)-2-chloro-Trityl Resin was obtained. The linker herein is 6-aminohexanoic acid, Ahx. TFA/TIS/H₂O=95/2.5/2.5 was added to the peptide made on the resin, and the conjugate was separated from the resin. A pre-chilled diethyl ether was added to the obtained mixture, and centrifugation was used to precipitate the peptide conjugates. After purification by Prep-HPLC, purity was confirmed with the analytical HPLC and the molecular weight was determined by LC/MS. The peptide synthesized as described above was verified as FITC-pep1 by confirmation of the molecular weight by LC/MS. Then the conjugates were lyophilized. SEQ ID NO: 1 to SEQ ID NO: 156 of peptides fused FITC with conjugate was also prepared as pep 1 of SEQ ID NO: 157.
(2) Preparation of a CPP-FITC Conjugate

The basic framework of the peptide, (NH₂-E(OtBu)-A-R(Pbf)-P-A-L-L-T(tBu)-S(tBu) -R(Pbf)L-R-(Pbf)-F-I-P-K(Dde)-2-chloro-Trityl Resin) was generated according to the manufacturing methods described in the Example 2 1. (1). To selectively introduce FITC to the C-term of the peptide, the N-term of the peptide was protected from Boc. Then, the D-tert-butyl dicarbonate (30 equivalent) and DIPEA (30 equivalent) were melted in DMF. The DMF solution was added to the peptide and incubated at room temperature for 2 hours, and the peptide was washed sequentially with DMF, MeOH, and DMF. As a result, Boc-E(OtBu)-A-R(Pbf)-P-A-L-L-T(tBu)-S(tBu)-R(Pbf)L-R(Pbf)-F-I-P-K(Dde)-2-chloro-Trityl Resin was obtained. Hydrazine in 2% of DMF was used to remove Dde which is the protecting group of the C-terminal residue Lys in order to add FITC to the C-terminal of Lys. Then, FITC (8 equivalent) and DIPEA (16 equivalent) were melted in DMF which was added to the peptide reaction mixture, and the mixture was incubated at room temperature for 2 hours, then washed sequentially with DMF, MeOH, DMF. As a result, Boc-E(OtBu)-A-R(Pbf)-P-A-L-L-T(tBu)-S(tBu)-R(Pbf)L-R(Pbf)-F-I-P-K(FITC) -2-chloro-Trityl Resin was obtained. TFA/TIS/H₂O=95/2.5/2.5 was added to separate the peptide from resin. Pre-chilled diethyl ether was added to the the mixture, and centrifugation was used to precipitate the peptides. After purification by Prep-HPLC, purity was confirmed with the analytical HPLC and the molecular weight was confirmed with LC/MS. The obtained substances were verified as pep1-FITC by confirmation of the molecular weight by LC/MS. Then the conjugates were lyophilized. SEQ ID NO: 1 to SEQ ID NO: 156 of peptide -FITC conjugates were also prepared in the same manner as described above pep1-FITC.

### Example 3: Experimental Cell Penetration of pep(CPP)-FITC Conjugate

### (1) Experimental Cell Penetration in HeLa Cell Line

### Cell Culture

Homo sapiens cervix adenocarcinoma cell line as HeLa cell lines were purchased from ATCC (American Type Cell Culture). The cells were cultured in MEM supplemented with 10% fetal bovine serum (Invitrogen, USA), Earle's salts, non-essential amino acids, sodium pyruvate and 100 μg/ml penicillin and 10 units/ml streptomycin and cultured at 37° C., 5% CO₂ incubator.

### Flow Cytometry and Confocal Microscoty Analysis of Cell Penetrating

Flow cytometry and Confocal microscope analysis were performed to compare the between degree of cellular uptake of the cells were treated with SEQ ID NO: 1 to SEQ ID NO: 156, pep (CPP) and control.

The cell line was divided in a 6-well plate and cultured in a medium containing 10% fetal bovine serum (Invitrogen, USA), 100 μg/ml penicillin, 100 units/ml streptomycin at 37° C., 5% CO₂ incubator for 12 hours. After washing the cell line with PBS, starvation was induced in a Minimum Essential Medium for an hour. 20 uM of each carrier peptide was treated and cultured at 37° C. for an hour. After repeating the step of washing the cells with PBS for three times, Trypsin-EDTA was treated form 10 mins at 37° C. to separate the carrier peptide on the outside of the cell. cells were collected with refrigerated PBS and centrifugation was performed to repeat the step of washing the cells for three times. After then, the cells were suspended in 0.5 ml of PBS containing 4% Paraformaldehyde and fluorescence of the cells was analyzed using FACS Calibur (Becton Dickinson). The cellular uptake aspect of control and various peptides combined with FITC was compared and analyzed by MFI (Mean Fluorescence Intensity).

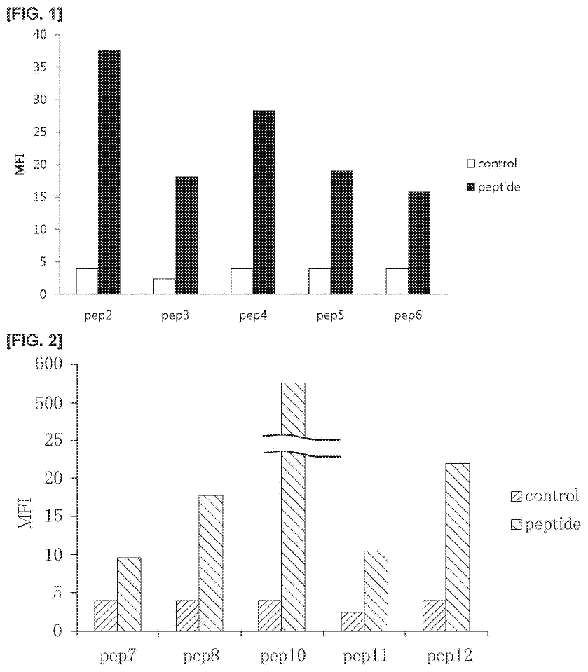

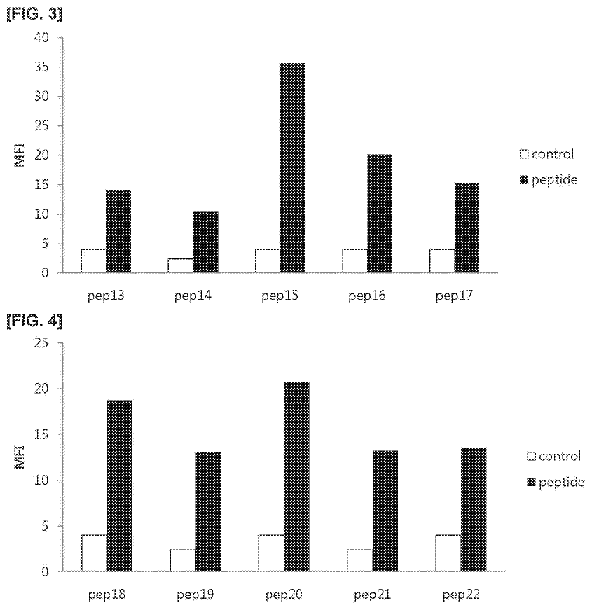

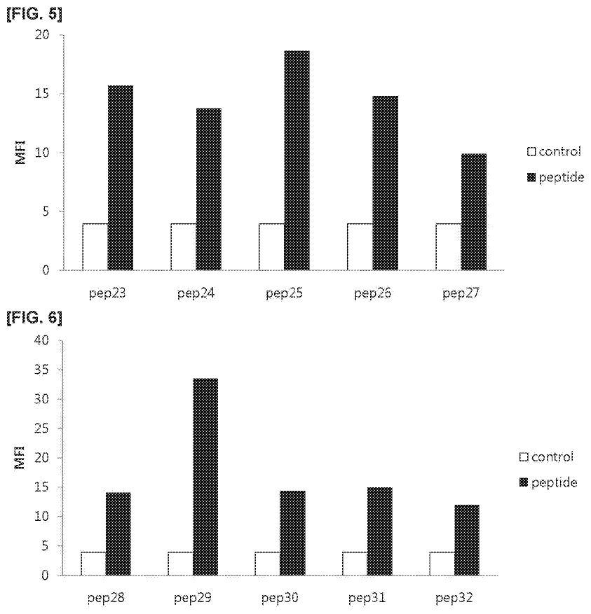

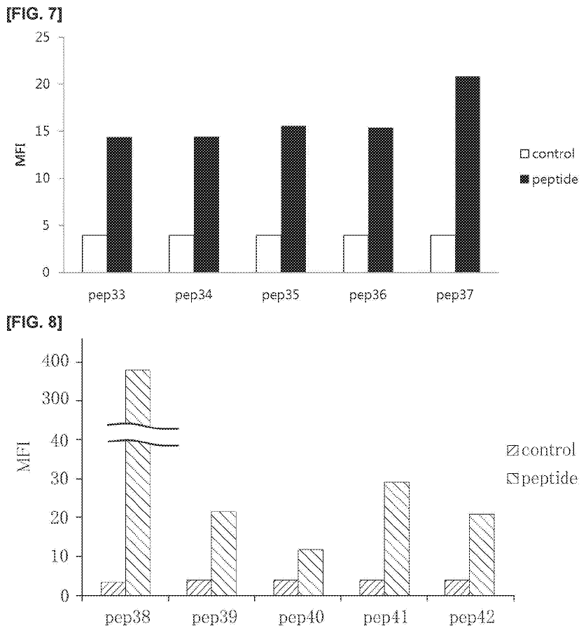

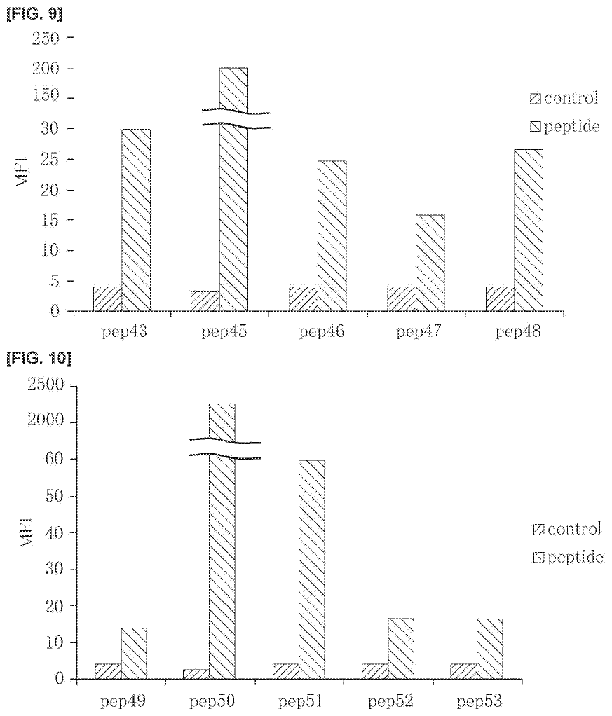

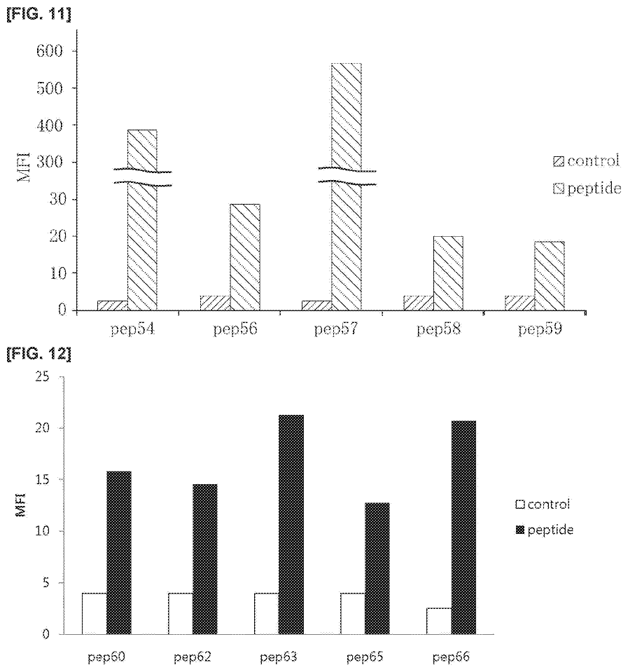

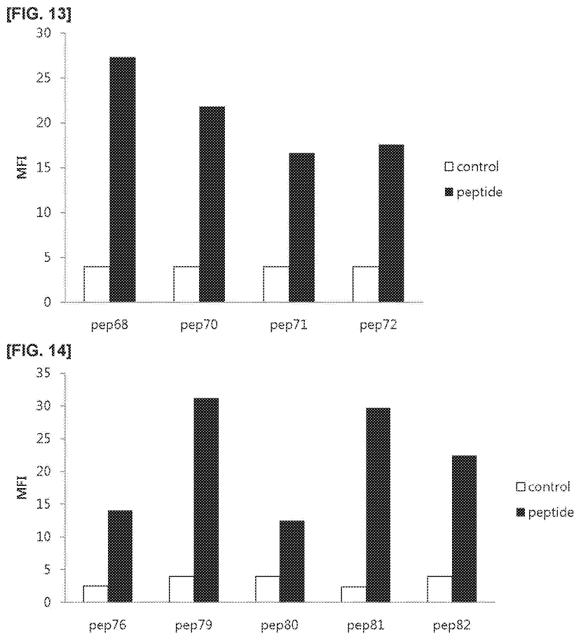

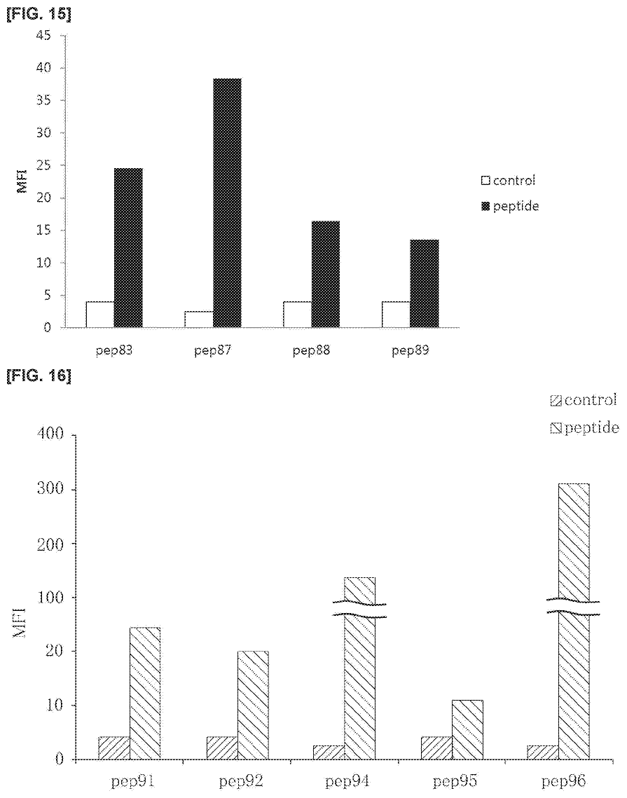

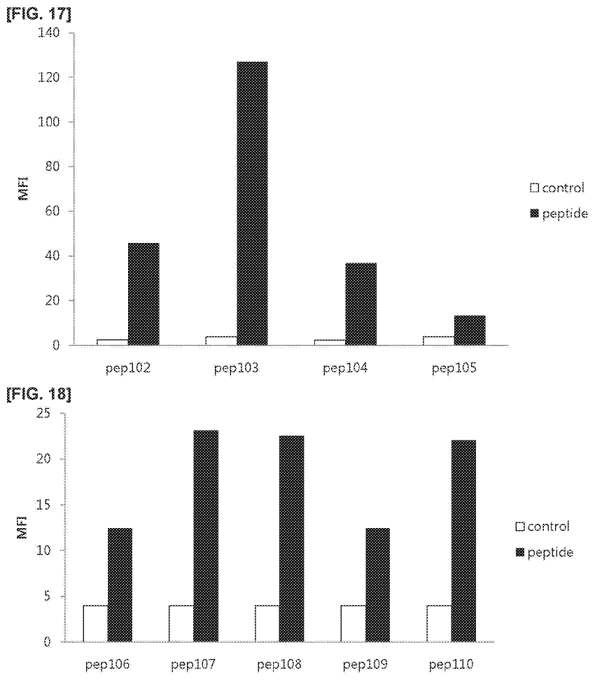

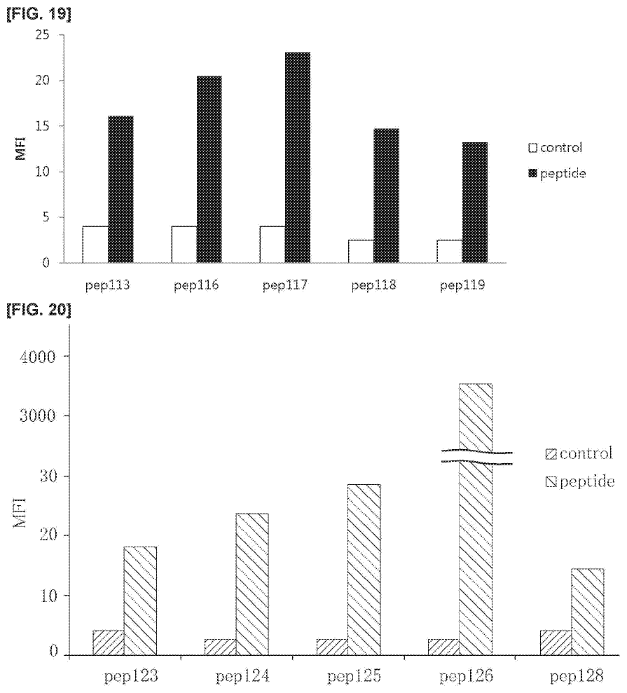

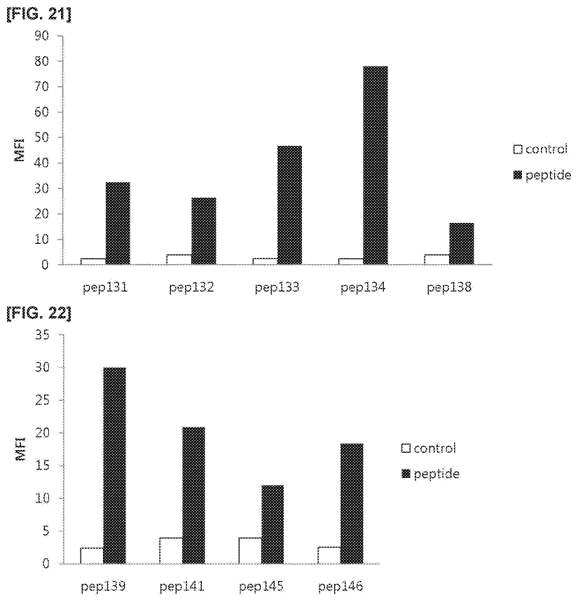

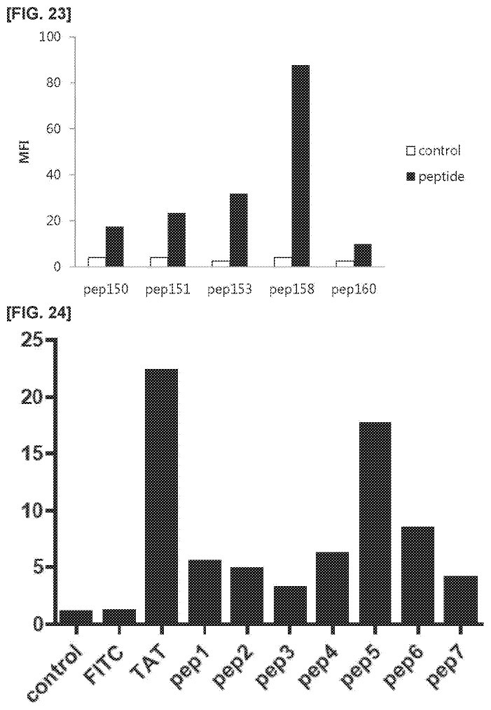

The results are as shown in FIGS. 1 to 23. Analysis results shown in FIG. 1 to FIG. 23 are given in detail in Table 3 below.

TABLE 3

| | pep2 | pep3 | pep4 | pep5 | pep6 | pep7 | pep8 |
|---|---|---|---|---|---|---|---|
| control | 3.98 ± 0.87 | 2.39 ± 0.23 | 3.98 ± 0.87 | 3.98 ± 0.87 | 3.98 ± 0.87 | 3.98 ± 0.87 | 3.98 ± 0.87 |
| peptide | 37.6 ± 1.89 | 18.23 ± 3.21 | 28.35 ± 2.54 | 19.06 ± 1.33 | 15.81 ± 1.59 | 9.51 ± 2.95 | 17.75 ± 1.96 |
| | pep10 | pep11 | pep12 | pep13 | pep14 | pep15 | pep16 |
| control | 3.98 ± 0.87 | 2.39 ± 0.23 | 3.98 ± 0.87 | 3.98 ± 0.87 | 2.39 ± 0.23 | 3.98 ± 0.87 | 3.98 ± 0.87 |
| peptide | 559.78 ± 3.41 | 10.46 ± 1.99 | 22 ± 2.6 | 14.01 ± 1.32 | 10.51 ± 3.71 | 35.61 ± 1.29 | 20.12 ± 1.84 |
| | pep17 | pep18 | pep19 | pep20 | pep21 | pep22 | pep23 |
| control | 3.98 ± 0.87 | 3.98 ± 0.87 | 2.39 ± 0.23 | 3.98 ± 0.87 | 2.39 ± 0.23 | 3.98 ± 0.87 | 3.98 ± 0.87 |
| peptide | 15.26 ± 2.23 | 18.7 ± 1.21 | 13.04 ± 3.41 | 20.73 ± 2.45 | 13.22 ± 1.67 | 13.56 ± 0.23 | 15.68 ± 2.03 |
| | pep24 | pep25 | pep26 | pep27 | pep28 | pep29 | pep30 |
| control | 3.98 ± 0.87 | 3.98 ± 0.87 | 3.98 ± 0.87 | 3.98 ± 0.87 | 3.98 ± 0.87 | 3.98 ± 0.87 | 3.98 ± 0.87 |
| peptide | 13.78 ± 1.07 | 18.62 ± 0.69 | 14.81 ± 1.77 | 9.89 ± 2.51 | 14.08 ± 3.11 | 33.5 ± 2.48 | 14.37 ± 1.73 |
| | pep31 | pep32 | pep33 | pep34 | pep35 | pep36 | pep37 |
| control | 3.98 ± 0.87 | 3.98 ± 0.87 | 3.98 ± 0.87 | 3.98 ± 0.87 | 3.98 ± 0.87 | 3.98 ± 0.87 | 3.98 ± 0.87 |
| peptide | 14.93 ± 2.05 | 11.98 ± 2.92 | 14.36 ± 1.63 | 14.41 ± 3.44 | 15.54 ± 1.38 | 15.37 ± 2.65 | 20.81 ± 1.02 |
| | pep38 | pep39 | pep40 | pep41 | pep42 | pep43 | pep45 |
| control | 3.31 ± 0.62 | 3.98 ± 0.87 | 3.98 ± 0.87 | 3.98 ± 0.87 | 3.98 ± 0.87 | 3.98 ± 0.87 | 3.31 ± 0.62 |
| peptide | 380.4 ± 2.6 | 21.4 ± 0.99 | 11.63 ± 1.11 | 28.97 ± 3.44 | 20.82 ± 1.93 | 29.88 ± 2.35 | 203.77 ± 3.26 |
| | pep46 | pep47 | pep48 | pep49 | pep50 | pep51 | pep52 |
| control | 3.98 ± 0.87 | 3.98 ± 0.87 | 3.98 ± 0.87 | 3.98 ± 0.87 | 2.39 ± 0.23 | 3.98 ± 0.87 | 3.98 ± 0.87 |
| peptide | 24.67 ± 2.02 | 15.85 ± 1.03 | 26.51 ± 2.36 | 13.91 ± 0.51 | 2228.76 ± 3.68 | 59.81 ± 1.38 | 16.67 ± 2.22 |
| | pep53 | pep54 | pep56 | pep57 | pep58 | pep59 | pep60 |
| control | 3.98 ± 0.87 | 2.52 ± 0.41 | 3.98 ± 0.87 | 2.39 ± 0.23 | 3.98 ± 0.87 | 3.98 ± 0.87 | 3.98 ± 0.87 |
| peptide | 16.16 ± 0.66 | 388.45 ± 2.94 | 28.61 ± 0.44 | 568.02 ± 3.01 | 19.87 ± 0.94 | 18.58 ± 0.52 | 15.79 ± 1.63 |
| | pep62 | pep63 | pep65 | pep66 | pep68 | pep70 | pep71 |
| control | 3.98 ± 0.87 | 3.98 ± 0.87 | 3.98 ± 0.87 | 2.52 ± 0.41 | 3.98 ± 0.87 | 3.98 ± 0.87 | 3.98 ± 0.87 |
| peptide | 14.55 ± 3.71 | 21.3 ± 1.55 | 12.72 ± 1.36 | 20.7 ± 2.57 | 27.28 ± 1.28 | 21.82 ± 1.76 | 16.61 ± 0.88 |
| | pep72 | pep76 | pep79 | pep80 | pep81 | pep82 | pep83 |
| control | 3.98 ± 0.87 | 2.52 ± 0.41 | 3.98 ± 0.87 | 3.98 ± 0.87 | 2.39 ± 0.23 | 3.98 ± 0.87 | 3.98 ± 0.87 |
| peptide | 17.57 ± 2.31 | 13.98 ± 0.11 | 31.2 ± 1.52 | 12.45 ± 0.46 | 29.69 ± 2.49 | 22.44 ± 3.32 | 24.57 ± 1.36 |
| | pep87 | pep88 | pep89 | pep91 | pep92 | pep94 | pep95 |
| control | 2.52 ± 0.41 | 3.98 ± 0.87 | 3.98 ± 0.87 | 3.93 ± 0.87 | 3.98 ± 0.87 | 2.39 ± 0.23 | 3.98 ± 0.87 |
| peptide | 38.35 ± 2.27 | 16.43 ± 0.55 | 13.54 ± 0.75 | 24.41 ± 2.79 | 19.91 ± 0.51 | 117.76 ± 4.24 | 10.92 ± 0.07 |
| | pep96 | pep102 | pep103 | pep104 | pep105 | prep106 | pep107 |
| control | 2.39 ± 0.23 | 2.52 ± 0.41 | 3.98 ± 0.87 | 2.39 ± 0.23 | 3.98 ± 0.87 | 3.98 ± 0.87 | 3.98 ± 0.87 |
| peptide | 308.53 ± 3.61 | 45.84 ± 1.58 | 126.88 ± 2.14 | 36.74 ± 1.11 | 13.23 ± 2.21 | 12.45 ± 0.38 | 23.11 ± 0.56 |
| | pep108 | pep109 | pep110 | pep113 | pep116 | pep117 | pep118 |
| control | 3.98 ± 0.87 | 3.98 ± 0.87 | 3.98 ± 0.87 | 3.98 ± 0.87 | 3.98 ± 0.87 | 3.98 ± 0.87 | 2.53 ± 0.17 |
| peptide | 22.53 ± 0.95 | 12.46 ± 1.32 | 22.04 ± 0.77 | 16.09 ± 2.5 | 20.5 ± 0.97 | 23.11 ± 2.46 | 14.69 ± 0.33 |
| | pep119 | pep123 | pep124 | pep125 | pep126 | pep128 | pep131 |
| control | 2.53 ± 0.17 | 3.98 ± 0.87 | 2.53 ± 0.17 | 2.53 ± 0.17 | 2.53 ± 0.17 | 3.98 ± 0.87 | 2.39 ± 0.23 |
| peptide | 13.23 ± 0.64 | 18.03 ± 2.83 | 23.74 ± 3.62 | 28.62 ± 2.12 | 3362.29 ± 3.24 | 14.36 ± 1.21 | 32.51 ± 2.38 |
| | pep132 | pep133 | pep134 | pep138 | pep139 | pep141 | pep145 |
| control | 3.98 ± 0.87 | 2.53 ± 0.17 | 2.39 ± 0.23 | 3.98 ± 0.87 | 2.39 ± 0.23 | 3.98 ± 0.87 | 3.98 ± 0.87 |
| peptide | 26.39 ± 0.95 | 46.79 ± 1.92 | 78.05 ± 2.26 | 16.43 ± 0.91 | 29.95 ± 0.28 | 20.89 ± 0.31 | 11.97 ± 0.78 |
| | pep146 | pep150 | pep151 | pep153 | pep158 | pep160 | |
| control | 2.53 ± 0.17 | 3.98 ± 0.87 | 3.98 ± 0.87 | 2.53 ± 0.17 | 3.98 ± 0.87 | 2.53 ± 0.17 | |
| peptide | 18.32 ± 1.82 | 17.46 ± 0.24 | 23.52 ± 0.56 | 31.72 ± 1.74 | 87.75 ± 2.51 | 9.860.23 | |

### (2) Cell Penetrating Property in Huh7 Cell Line

### Cell Culture

human hepatocellular carcinoma cell line as Huh7 lines were purchased from ATCC (American Type Cell Culture) and used as suspended cells. The cells were cultured in MEM supplemented with 10% fetal bovine serum (Invitrogen, USA), EarleSA), Earle Huh7 lines were purchased from ATpyruvate and 100 μa/ml penicillin and 10 units/ml streptomycin and cultured at 37° C., 5% CO₂ incubator.

### The Screening Analysis of Cell Penetrating Used by Flow Cytometry

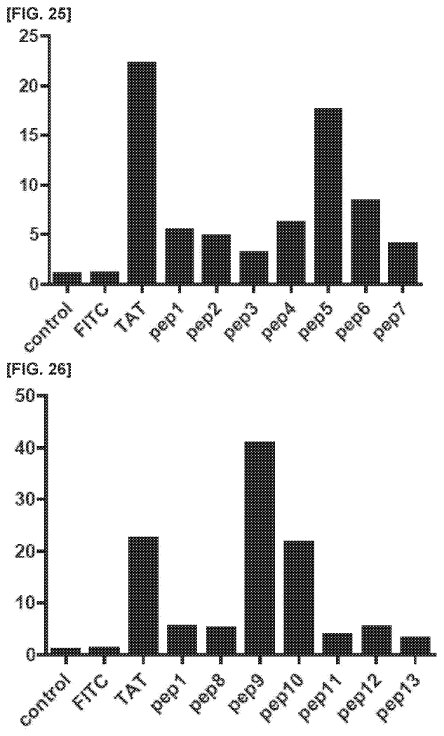

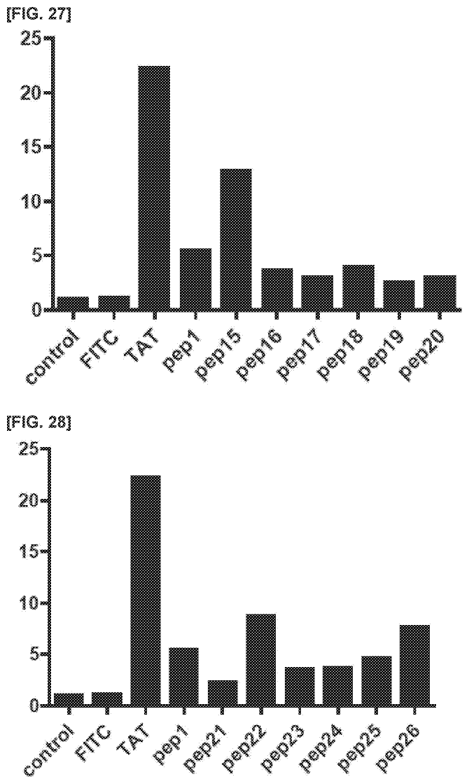

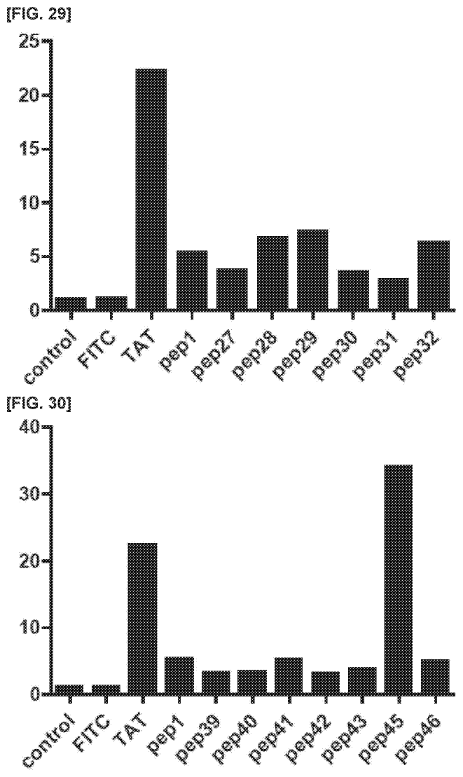

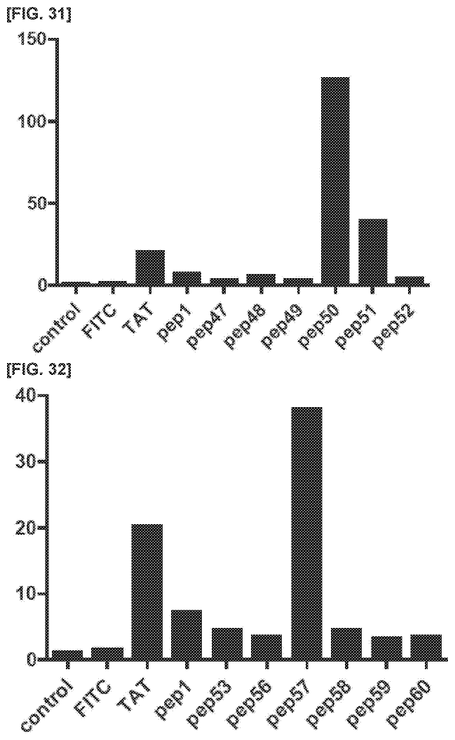

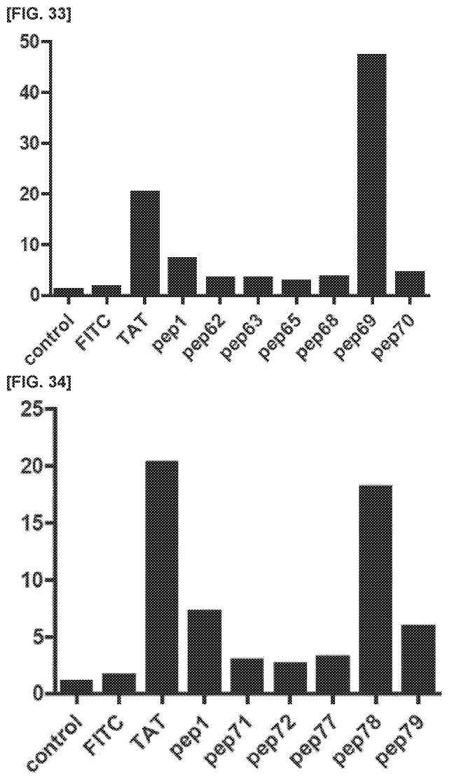

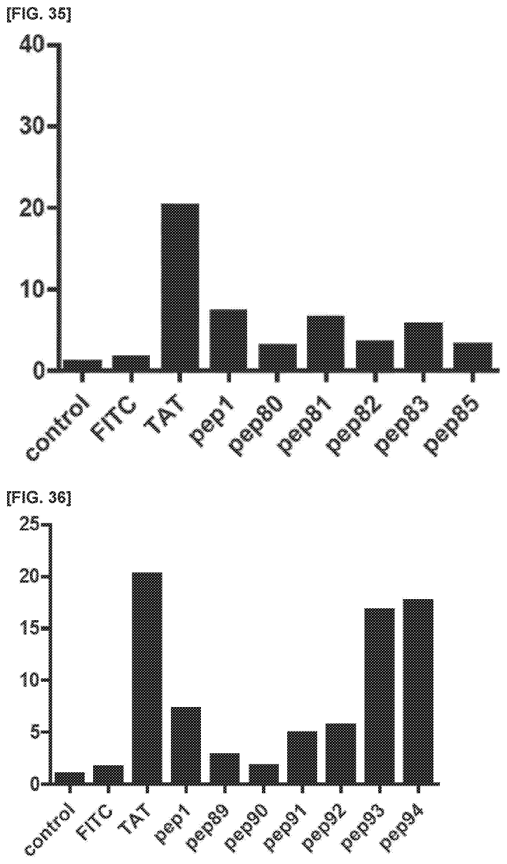

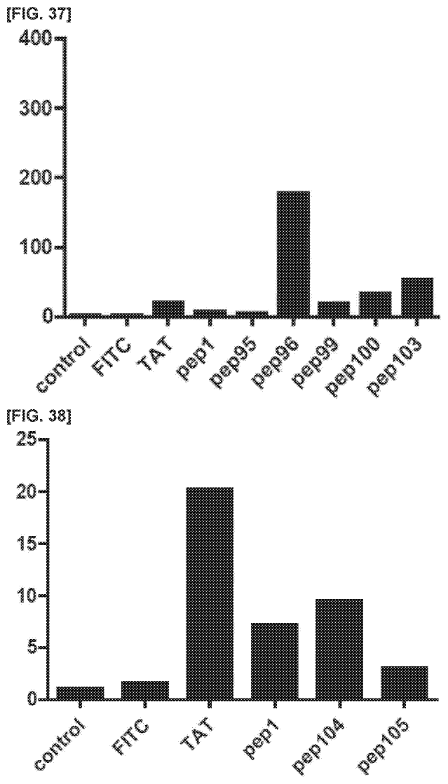

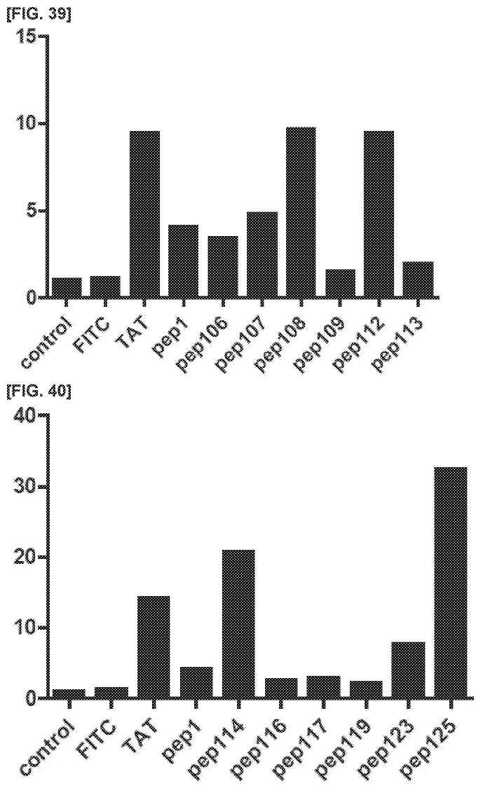

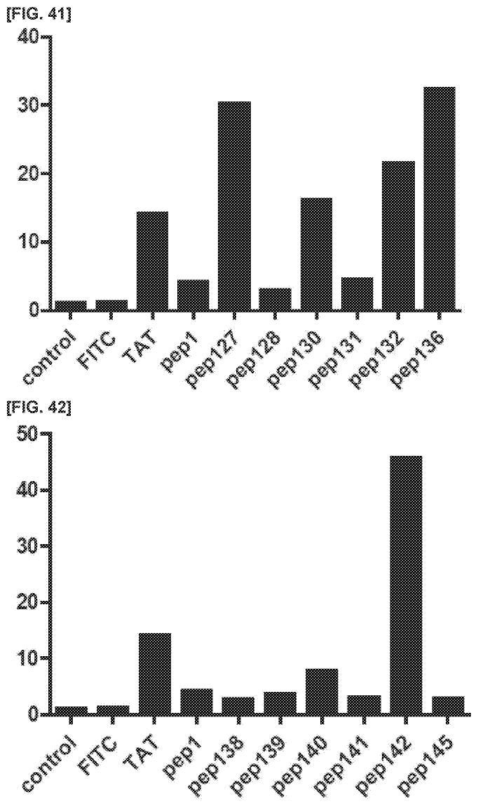

To confirm the cell penetrating of peptides, the Huh7 cell lines were treated with SEQ ID NO: 1 to SEQ ID NO: 156 and analysed by Flow cytometry. The analysed method was also confirmed by the same manner as described above example (1) in Hela cell lines, Also, the result showed in FIG. 24 to FIG. 43.

### (3) Experimental Cell Penetration in Human T Lymphocyte Cell Lines

### Cell Culture

human T-cell leukemia cell line as Jurket was purchased from ATCC (American Type Cell Culture) and used as suspended cells. The cells were cultured in RPMI 1640 supplemented with 10% fetal bovine serum (Invitrogen, USA), EarleSA), EarleC (American Type Cell Culture) and pyruvate and 100 om ATCC (American Type units/ml streptomycin and cultured at 37° C., 5% CO₂ incubator. Human derived lymphocytes (lymphocyte) separated from the healthy human blood (50 m) and then collected layer of peripheral blood mononuclear cells (PBMC) and lymphocytes used by Biocoll separating solution (Biochrom AG, Berlin, Germany).

### The Screening Analysis of Cell Penetrating Used by Flow Cytometry

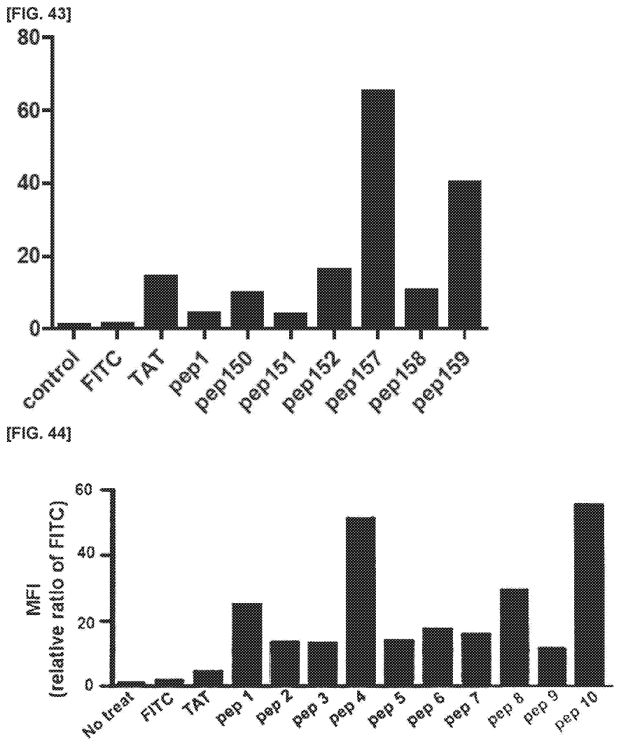

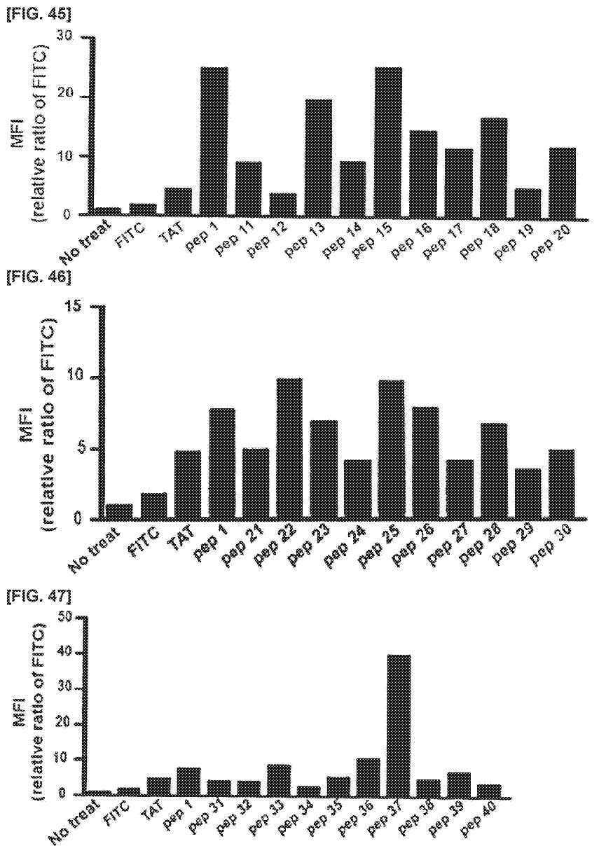

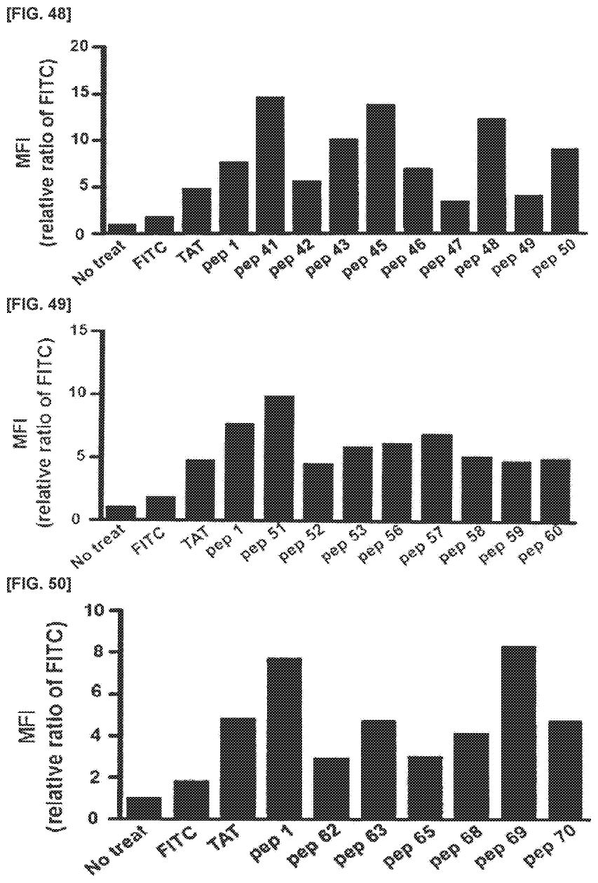

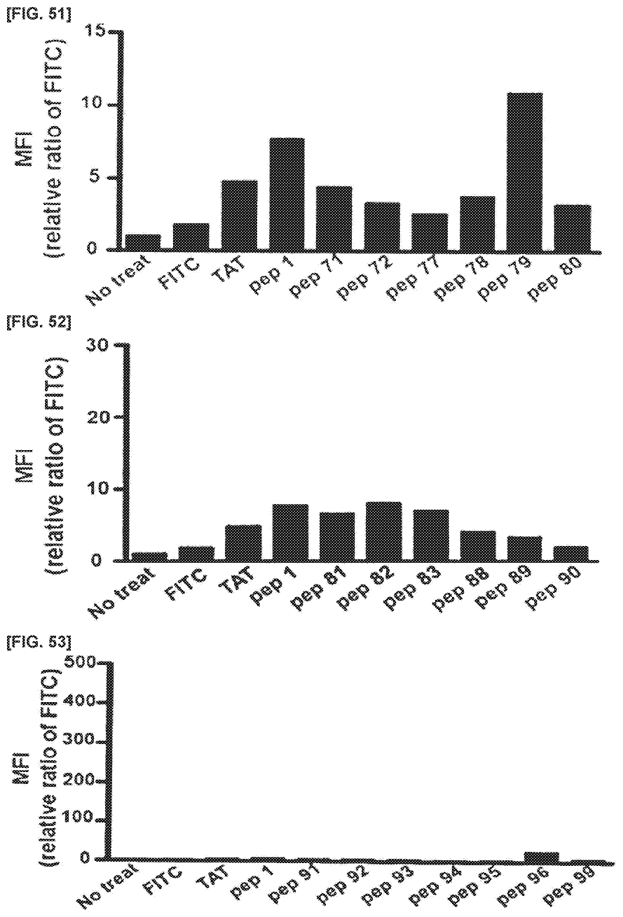

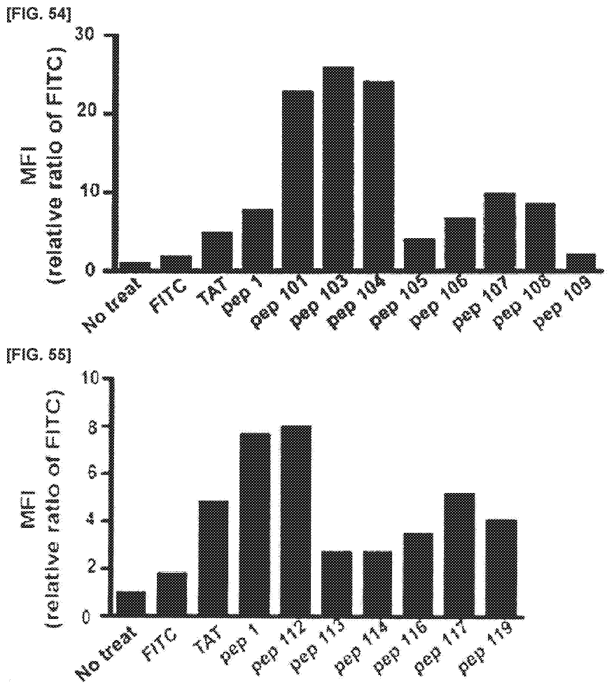

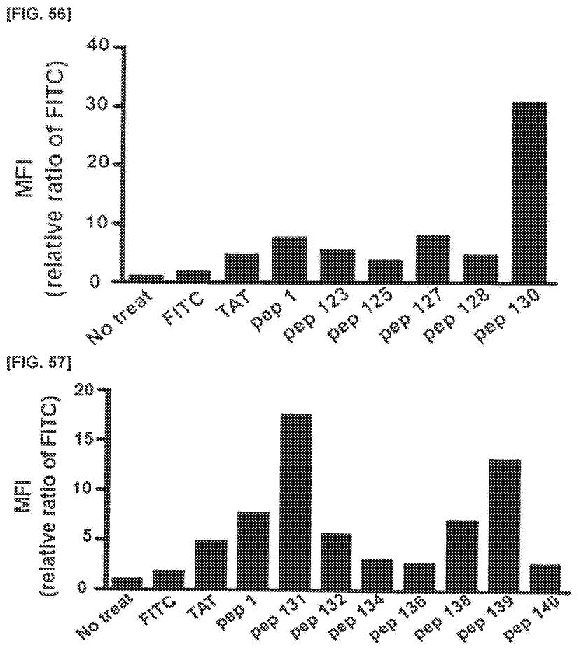

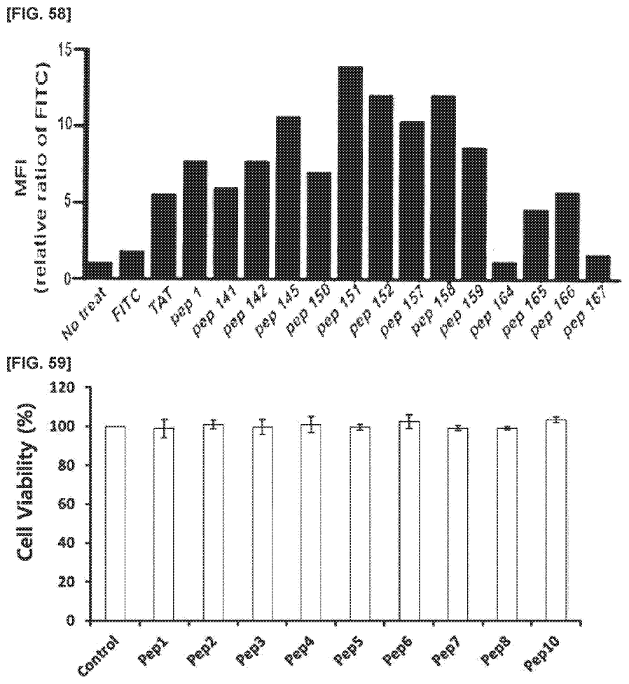

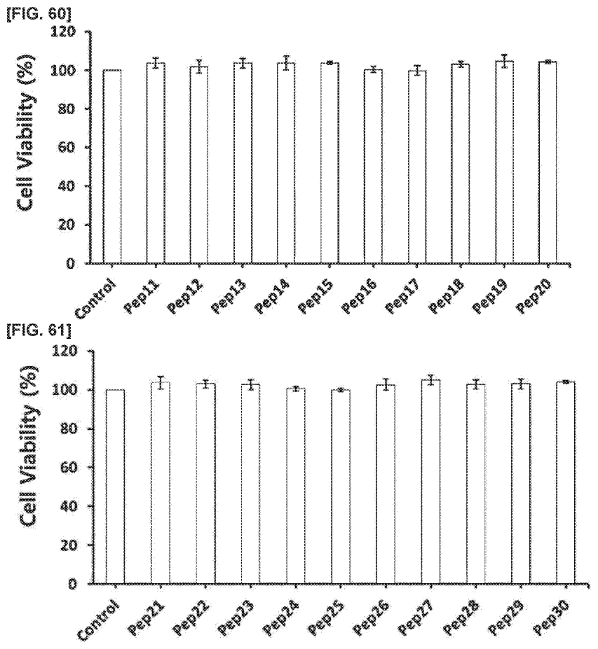

To confirm the cell penetrating of peptides, the human T-cell leukemia cell lines were treated with SEQ ID NO: 1 to SEQ ID NO: 156 and analysed by Flow cytometry. The analysed method was also confirmed by the same manner as described above example (1) Hela cell lines. Also, the result showed in FIG. 44 to FIG. 58.

### (4) Analysis of Cell Viability and Toxicity

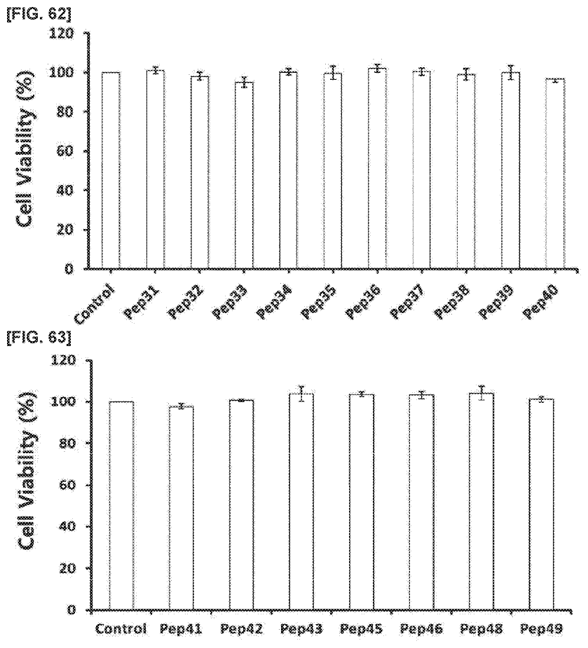

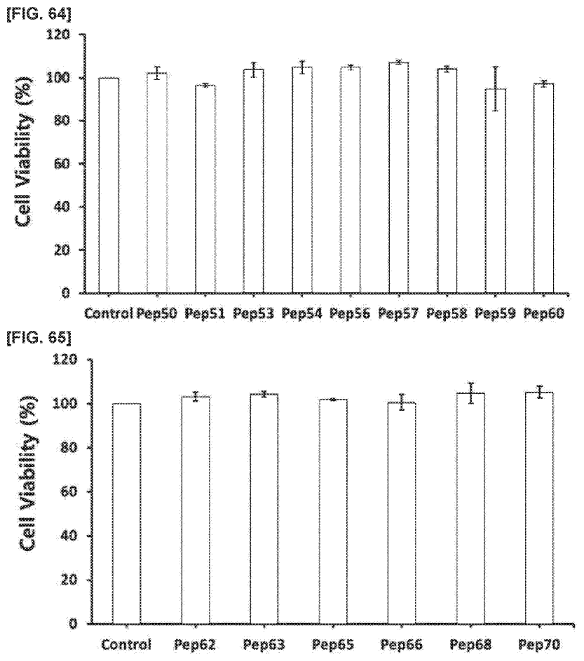

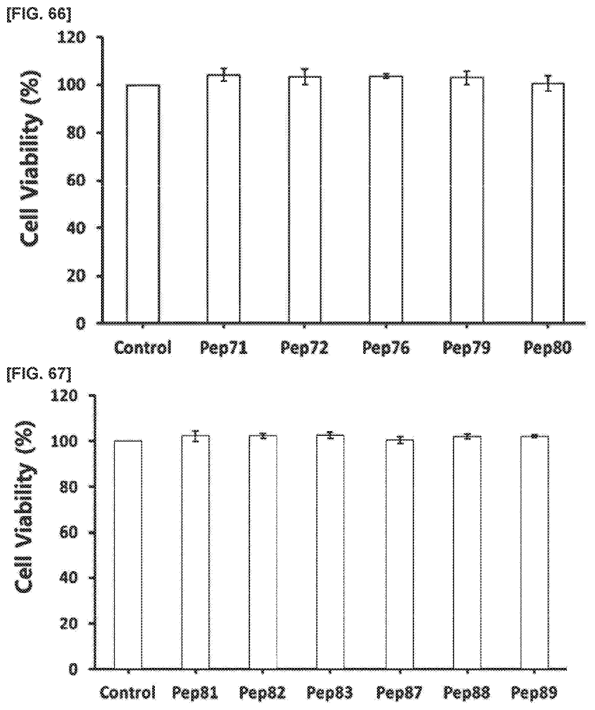

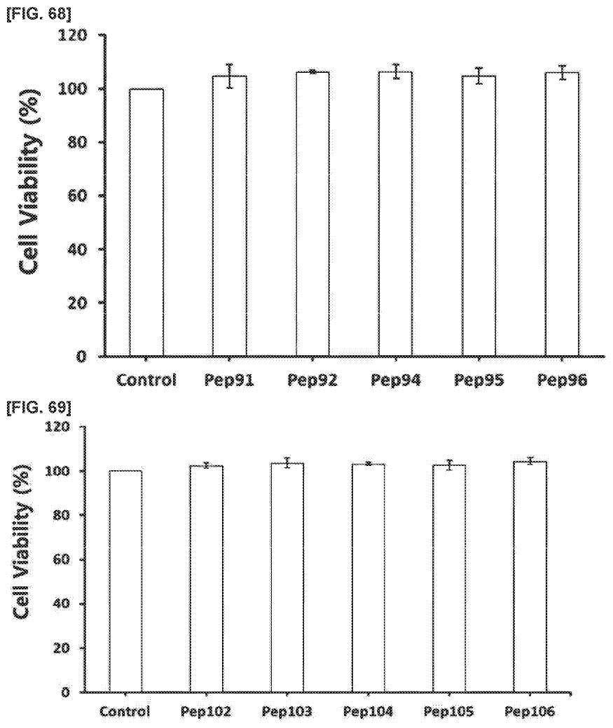

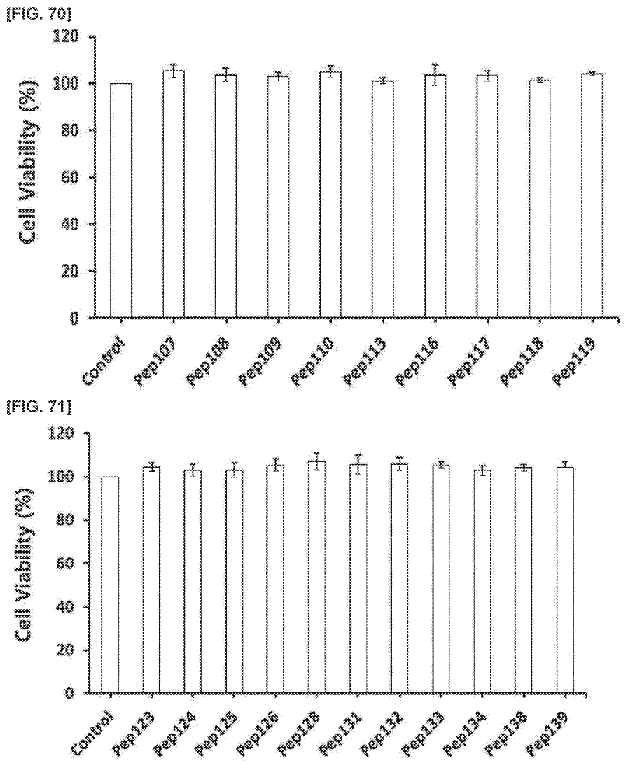

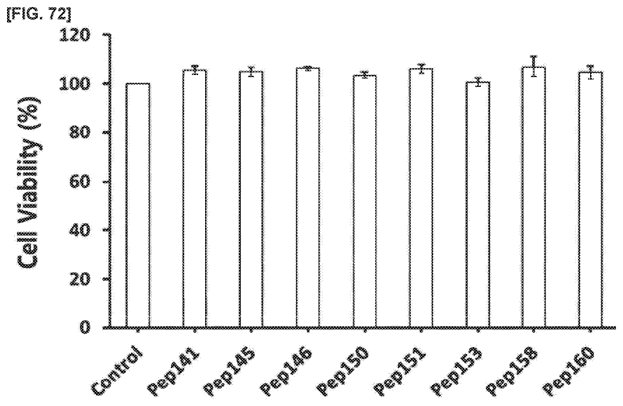

The cell line was divided into 96-well plate and cultured in a medium containing 10% fetal bovine serum (Invitrogen, USA), 100 μg/ml penicillin, 100 units/ml streptomycin at 37° C., 5% CO₂ incubator for 12 hours. After washing the cell line with PBS, starvation was induced in a Minimum Essential Medium for an hour. 20 uM of each carrier peptide was treated and cultured at 37° C. for an hour. After cultured cells, analysed cell viability and toxicity used by MTT assay. The results showed in FIG. 59 to FIG. 72.

1-27. (canceled)

28. A conjugate of a cell penetrating carrier peptide and an active ingredient, wherein the carrier peptide comprises of any one amino acid sequence of SEQ ID NO: 98, SEQ ID NO: 153, SEQ ID NO: 1 to SEQ ID NO: 8, SEQ ID NO: 10 to SEQ ID NO: 47, SEQ ID NO: 49 to SEQ ID NO: 78, SEQ ID NO: 80 to SEQ ID NO: 97, SEQ ID NO: 99 to SEQ ID NO: 120, SEQ ID NO: 122 to SEQ ID NO: 134, SEQ ID NO:136 to SEQ ID NO: 152, and SEQ ID NO: 154 to SEQ ID NO: 156, the peptide having above 80% homology with the above-mentioned peptide sequences, or the fragment of the above mentioned peptides, and wherein the peptide having above 80% homology with the sequence, and the fragment are the peptides that maintain cell penetrating ability of any one amino acid sequence of SEQ ID NO: 98, SEQ ID NO: 153, SEQ ID NO: 1 to SEQ ID NO: 8, SEQ ID NO: 10 to SEQ ID NO: 47, SEQ ID NO: 49 to SEQ ID NO: 78, SEQ ID NO: 80 to SEQ ID NO: 97, SEQ ID NO: 99 to SEQ ID NO: 120, SEQ ID NO: 122 to SEQ ID NO: 134, SEQ ID NO:136 to SEQ ID NO: 152, and SEQ ID NO: 154 to SEQ ID NO: 156.

29. The conjugate according to claim 28, wherein the fragment is made of at least 3 amino acids.

30. The conjugate according to claim 28, wherein the carrier peptide is made of 30 or less amino acids.

31. The conjugate according to claim 28, wherein the active ingredient is at least one selected from protein, nucleic acid, peptide, lipid, glycol-lipid, mineral, sugar, nanoparticle, biological products, contrast agent, drugs, and chemical compounds.

32. The conjugate according to claim 28, wherein the carrier peptide and the active ingredient are combined via a covalent bond, selectively mediated by a linker.

33. The conjugate according to claim 28, wherein the carrier peptide and the active ingredient are combined via non-covalent bond.

34. The conjugate according to claim 31, wherein the active ingredient is a cytokine, antibody, fragment of antibody, therapeutic enzyme, soluble receptor, or ligand.

35. The conjugate according to claim 31, wherein the contrast agent is selected from the group consisting of radiopaque contrast agent, paramagnetic contrast agent, superparamagnetic contrast agent, and CT contrast agent.

36. The conjugate according to claim 31, wherein the contrast agent is based on iron.

37. The conjugate according to claim 36, wherein the contrast agent is a ferrocene carboxylate.

38. A contrast agent comprising a conjugate of claim 28.

39. A composition comprising the conjugate according to claim 28 as an active ingredient.

40. The composition according to claim 39, wherein the active ingredient is selected from the group consisting of an active agent for treatment or prevention of disease, an active ingredient of functional cosmetics, and an active ingredient of health functional food; and wherein the composition is selected from the group consisting of a pharmaceutical composition, a cosmetic composition, and a health food composition.

41. A cell penetrating carrier peptide, wherein the carrier peptide comprises of any one amino acid sequence of SEQ ID NO: 98, SEQ ID NO: 153, SEQ ID NO: 1 to SEQ ID NO: 8, SEQ ID NO: 10 to SEQ ID NO: 47, SEQ ID NO: 49 to SEQ ID NO: 78, SEQ ID NO: 80 to SEQ ID NO: 97, SEQ ID NO: 99 to SEQ ID NO: 120, SEQ ID NO: 122 to SEQ ID NO: 134, SEQ ID NO:136 to SEQ ID NO: 152, and SEQ ID NO: 154 to SEQ ID NO: 156, the peptide having above 80% homology with the above-mentioned peptide sequences, or the fragment of the above mentioned peptides.

42. A polynucleotide that encodes the peptide according to claim 41.

43. A vector that comprises the polynucleotide according to claim 42.

44. A transformed cell that comprises the vector according to claim 43.

45. A method of treatment a subject having a mitochondrial-related disease or disorder comprising administering to the subject a pharmaceutical composition comprising a conjugate of a cell penetrating carrier peptide and an active ingredient, wherein the carrier peptide comprises of any one amino acid sequence of SEQ ID NO: 98, SEQ ID NO: 153, SEQ ID NO: 1 to SEQ ID NO: 8, SEQ ID NO: 10 to SEQ ID NO: 47, SEQ ID NO: 49 to SEQ ID NO: 78, SEQ ID NO: 80 to SEQ ID NO: 97, SEQ ID NO: 99 to SEQ ID NO: 120, SEQ ID NO: 122 to SEQ ID NO: 134, SEQ ID NO:136 to SEQ ID NO: 152, and SEQ ID NO: 154 to SEQ ID NO: 156, the peptide having above 80% homology with the above-mentioned peptide sequences, or the fragment of the above mentioned peptides, and wherein the peptide having above 80% homology with the sequence, and the fragment are the peptides that maintain cell penetrating ability of any one amino acid sequence of SEQ ID NO: 98, SEQ ID NO: 153, SEQ ID NO: 1 to SEQ ID NO: 8, SEQ ID NO: 10 to SEQ ID NO: 47, SEQ ID NO: 49 to SEQ ID NO: 78, SEQ ID NO: 80 to SEQ ID NO: 97, SEQ ID NO: 99 to SEQ ID NO: 120, SEQ ID NO: 122 to SEQ ID NO: 134, SEQ ID NO:136 to SEQ ID NO: 152, and SEQ ID NO: 154 to SEQ ID NO: 156.

46. The method of claim 45, wherein the pharmaceutical composition is administered though oral, rectal, transdermal, intravenous, intramuscular, intraperitoneal, in the bone marrow, epidural, or subcutaneous means.

47. The method of claim 45, wherein the pharmaceutical composition comprises additives selected from the group consisting of diluents, excipients, lubricants, binders, disintegrants, buffers, dispersants, surfactants, coloring agents, aromatics, and sweeteners.
